(19) United States
(12) Patent Application Publication
(10) Pub. No.: US 2023/0062192 A1
(43) Pub. Date: Mar. 2, 2023

(54) SYSTEMS AND METHODS FOR MITIGATING TEMPERATURE-BASED NOISE IN ELECTROPHYSIOLOGICAL INFORMATION

(71) Applicant: KARDIUM INC., Burnaby (CA)

(72) Inventors: Mohammad Hassan SHARIAT, Vancouver (CA); Daniel Martin REINDERS, Richmond (CA); Justin Aaron MICHAEL, Vancouver (CA)

(21) Appl. No.: 17/886,045

(22) Filed: Aug. 11, 2022

Related U.S. Application Data
(60) Provisional application No. 63/238,695, filed on Aug. 30, 2021.

(51) Int. Cl. A61B 18/14

(52) U.S. Cl. CPC A61B 18/1492; A61B 2018/00577

(57) ABSTRACT

An electrophysiological information processing system may be configured to acquire electrophysiological information indicating sensed electrophysiological activity produced within a patient body, the electrophysiological activity sensed via at least a first transducer. Temperature information may be received indicating temperature at least proximate the first transducer. The electrophysiological information may be modified in accordance with the received temperature information, and the modified electrophysiological information may be output.

### CROSS-REFERENCE TO RELATED APPLICATION

This application claims the benefit of U.S. Provisional Application No. 63/238,695, filed Aug. 30, 2021, the entire disclosure of which is hereby incorporated herein by reference.

### TECHNICAL FIELD

Aspects of this disclosure generally are related to systems and methods for reducing temperature-based noise in sensed electrophysiological information.

### BACKGROUND

Various transducer-based devices are employed to measure electrophysiological potential on or within a patient's body, such as inside of the heart. For example, in various cardiac applications, the measurement of electrophysiological potential may assist a health care practitioner in evaluating the health or condition of a portion of the heart, or guide the health care practitioner in the performance of various cardiac therapies such as cardiac ablation, electrical stimulation therapy, or evaluate the efficacy of cardiac therapy. An electrocardiogram (ECG or EKG) measures the electrical activity of the heart with electrodes positioned on an external (e.g., skin) surface of a body. An electrogram is a representation of electrical activity of an organ such as the heart or brain via electrode positioned on or in the tissue rather than on an external surface of the body.

Electrophysiological activity generally produces low amplitude signals that are typically recorded in the presence of numerous sources of noise and interference. Conventional techniques view this noise as constant or non-time-varying (e.g., “DC”) bias voltage. As a result, it is common to use common-mode noise rejection or a high-pass filter with a low cut-off frequency (e.g., 0.05 Hz) to remove the DC bias from the measured electrophysiological voltage signals.

The present inventors, however, have realized that some noise associated with the measured electrophysiological voltage signals may be non-constant or time-varying in nature. For example, the present inventors have determined that temperature-dependent effects may be a source of, or a contributor of time-varying noise in the measured electrophysiological voltage signals. The present inventors have determined that, when temperature changes over time, those temperature-dependent effects are not stable, which, in turn, leads to non-DC effects that are not removed with standard techniques.

In this regard, the present inventors have recognized that there is a need in the art for systems and methods for reducing the deleterious effects associated with the presence of time-varying noise on measured electrophysiological voltage signals. The present inventors also have recognized that there is a need in the art for systems and methods for reducing the deleterious effects associated with temperature-dependent effects on measured electrophysiological voltage signals.

### SUMMARY

At least the above-discussed need is addressed and technical solutions are achieved in the art by various embodiments of the present invention.

According to some embodiments, an electrophysiological information processing system may be summarized as including an input-output device system, a memory device system storing a program, and a data processing device system communicatively connected to the input-output device system and the memory device system. In some embodiments, the data processing device system may be configured at least by the program at least to receive electrophysiological information indicating sensed electrophysiological activity produced within a patient body, the electrophysiological activity sensed via at least a first transducer. In some embodiments, the data processing device system may be configured at least by the program at least to receive, via the input-output device system, temperature information indicating temperature at least proximate the first transducer. In some embodiments, the data processing device system may be configured at least by the program at least to modify the received electrophysiological information in accordance with the received temperature information. In some embodiments, the data processing device system may be configured at least by the program at least to cause output, via the input-output device system, of the modified electrophysiological information.

In some embodiments, the input-output device system includes a transducer-based device, the transducer-based device configured to deliver at least the first transducer to a bodily cavity inside the patient body. In some embodiments, the received electrophysiological information may indicate the sensed electrophysiological activity produced within the patient body at least during a first state in which at least the first transducer is in the bodily cavity inside the patient body. In some embodiments the received temperature information may indicate the temperature at least proximate the first transducer at least during the first state in which at least the first transducer is in the bodily cavity inside the patient body.

In some embodiments, the first transducer may be configured to sense temperature, and the temperature information may indicate the temperature at least proximate the first transducer is sensed via the first transducer. In some embodiments, the first transducer may include an electrode configured to sense the electrophysiological activity, and may include a thermal sensor responsive to the temperature at least proximate the first transducer.

In some embodiments, the data processing device system may be configured at least by the program at least to cause at least part of the transducer-based device to transmit energy. In some embodiments, the received temperature information indicating the temperature at least proximate the first transducer may be responsive to the energy transmitted by the at least part of the transducer-based device in the bodily cavity at least during the first state in which at least the first transducer is in the bodily cavity inside the patient body. In some embodiments, the part of the transducer-based device may include a second transducer configured to transmit the energy. In some embodiments, the energy may be sufficient to ablate tissue in the bodily cavity. In some embodiments, the energy may be configured to thermally ablate tissue in the bodily cavity.

In some embodiments, the first transducer may be configured to transmit tissue ablative energy. In some embodiments, the first transducer may include a first electrode including a first conductive metal. In some embodiments, the input-output device system may include a second electrode including a second metal. In some embodiments, the received electrophysiological information indicating the sensed electrophysiological activity produced within the patient body may be based at least on intracardiac voltage data indicating electric potential between the first electrode and the second electrode. In some embodiments, the second electrode may be configured to be externally applied to the patient body comprising the bodily cavity. In some embodiments, the data processing device system may be configured at least by the program at least to produce the modified electrophysiological information in accordance with the received temperature information to mitigate noise in at least the intracardiac voltage data caused by thermoelectric effects during measurement of the electric potential between the first electrode and the second electrode. In some embodiments, the data processing device system may be configured at least by the program at least to produce the modified electrophysiological information in accordance with the received temperature information to mitigate noise in at least the intracardiac voltage data caused at least in part by a temperature difference between the first electrode and the second electrode during measurement of the electric potential between the first electrode and the second electrode. In some embodiments, the second metal may be different than the first metal. In some embodiments, the data processing device system may be configured at least by the program at least to produce the modified electrophysiological information in accordance with the received temperature information to mitigate noise in at least the intracardiac voltage data caused by temperature-dependent galvanic cell potential effects during measurement of the electric potential between the first electrode and the second electrode.

In some embodiments, the temperature information may indicate temperature at least proximate the first transducer during a time interval. In some embodiments, the data processing device system may be configured at least by the program at least to, in accordance with the received temperature information, (a) determine an amount of change in temperature at least proximate the first transducer, and (b) determine an amount of modification of the received electrophysiological information based at least on the determined amount of change in temperature at least proximate the first transducer. In some embodiments, the data processing device system may be configured at least by the program at least to produce the modified electrophysiological information in accordance with the received temperature information in accordance with the determined amount of modification. In some embodiments, the electrophysiological information may indicate the sensed electrophysiological activity produced within the patient body during the time interval.

In some embodiments, the data processing device system may be configured at least by the program at least to, in accordance with the received temperature information, (a) determine an amount of change in temperature at least proximate the first transducer, and (b) determine an amount of modification of the received electrophysiological information based at least on the determined amount of change in temperature at least proximate the first transducer. In some embodiments, the data processing device system may be configured at least by the program at least to produce the modified electrophysiological information at least in part by subtracting the determined amount of modification from the received electrophysiological information.

In some embodiments, the temperature information may indicate temperature at least proximate the first transducer during a time interval. In some embodiments, the received electrophysiological information may indicate an electrophysiological information waveform representing the sensed electrophysiological activity produced within the patient body and sensed via at least the first transducer during the time interval. In some embodiments, the data processing device system may be configured at least by the program at least to generate a temperature-based information waveform representing the temperature at least proximate the first transducer during the time interval indicated by the temperature information. In some embodiments, the data processing device system may be configured at least by the program at least to produce the modified electrophysiological information at least in part by subtracting the temperature-based information waveform from the electrophysiological information waveform.

In some embodiments, the data processing device system may be configured at least by the program at least to determine the amount of the modification based at least in part via a linear mapping of the temperature information. In some embodiments, the data processing device system may be configured at least by the program at least to determine the amount of the modification based at least in part via a linear mapping over time of the temperature information.

In some embodiments, an electrophysiological information processing system may include an input-output device system, a memory device system storing a program, and a data processing device system communicatively connected to the input-output device system and the memory device system, the data processing device system configured at least by the program at least to: acquire electrophysiological information indicating sensed electrophysiological activity produced in a patient body, the electrophysiological activity sensed via at least a first transducer; acquire temperature information indicating temperature change at least proximate the first transducer; modify the electrophysiological information in accordance with the acquired temperature information; and cause output, via the input-output device system, of the modified electrophysiological information.

Combinations and sub-combinations of the systems described above may form other systems according to various embodiments.

Various embodiments of the present invention may include systems, devices, or machines that are or include combinations or subsets of any one or more of the systems, devices, or machines and associated features thereof summarized above or otherwise described herein (which should be deemed to include the figures).

Further, all or part of any one or more of the systems, devices, or machines summarized above or otherwise described herein or combinations or sub-combinations thereof may implement or execute all or part of any one or more of the processes or methods described herein or combinations or sub-combinations thereof.

For example, according to some embodiments, a method of processing electrophysiological information is provided. In some embodiments, the method may be executed by a data processing device system configured by a program, the data processing device system communicatively connected to an input-output device system and a memory device system, the memory device system storing the program. In some embodiments, the method may include acquiring electrophysiological information indicating sensed electrophysiological activity produced in a patient body, the electrophysiological activity sensed via at least a first transducer. In some embodiments, the method may include acquiring temperature information indicating temperature change at least proximate the first transducer. In some embodiments, the method may include modifying the electrophysiological information in accordance with the acquired temperature information. In some embodiments, the method may include causing output, via the input-output device system, of the modified electrophysiological information.

In some embodiments, the electrophysiological activity may be sensed via at least the first transducer at least during a first state in which the first transducer is in a bodily cavity in the patient body. In some embodiments, the method may include causing, via a transducer-based device communicatively connected to the input-output device system, transmission of energy in the bodily cavity in the patient body at least during the first state in which the first transducer is in the bodily cavity in the patient body. In some embodiments, the temperature change at least proximate the first transducer may be responsive to the transmitted energy. In some embodiments, the transmitted energy may be sufficient to cause tissue ablation. In some embodiments, the method may include causing, via the transducer-based device communicatively connected to the input-output device system, radiofrequency (“RF”) ablation of tissue in the bodily cavity in the patient body. In some embodiments, the energy may be transmitted at least as part of the causing RF ablation of tissue in the bodily cavity in the patient body. In some embodiments, the method may include causing, via the transducer-based device communicatively connected to the input-output device system, pulsed field ablation of tissue in the bodily cavity in the patient body. In some embodiments, the energy may be transmitted at least as part of the causing pulsed field ablation of tissue in the bodily cavity in the patient body. In some embodiments, the method may include causing, via a cryogenic device communicatively connected to the input-output device system, cryoablation of tissue in the bodily cavity in the patient body. In some embodiments, the energy may be transmitted at least as part of the causing cryoablation of tissue in the bodily cavity in the patient body.

In some embodiments, the transducer-based device may be delivered into the bodily cavity in the patient body. In some embodiments, the method may include determining a level of contact between at least part of the transducer-based device and a tissue surface of the bodily cavity in the patient body, wherein the energy is transmitted at least as part of the determining the level of contact between at least part of the transducer-based device and the tissue surface of the bodily cavity in the patient body. In some embodiments, a fluid may be delivered to the bodily cavity in the patient body, and the temperature change at least proximate the first transducer is responsive to the delivered fluid.

In some embodiments, the modifying the electrophysiological information may include determining an amount of the temperature change at least proximate the first transducer. In some embodiments, the modifying the electrophysiological information may include determining an amount of modification of the acquired electrophysiological information based at least on the determined amount of the temperature change at least proximate the first transducer. In some embodiments, the modifying the electrophysiological information may include producing the modified electrophysiological information in accordance with the determined amount of modification. In some embodiments, the method may include determining the amount of the modification at least in part via a linear mapping over time of the temperature information.

In some embodiments, the temperature information may indicate temperature change during a time interval. In some embodiments, the electrophysiological activity produced in the patient body may be sensed during the time interval.

In some embodiments, a method of processing electrophysiological information is provided, the method executed by a data processing device system configured by a program, the data processing device system communicatively connected to an input-output device system and a memory device system, the memory device system storing the program. In some embodiments, the method includes receiving electrophysiological information indicating sensed electrophysiological activity produced within a patient body, the electrophysiological activity sensed via at least a first transducer; receiving, via the input-output device system, temperature information indicating temperature at least proximate the first transducer; modifying the received electrophysiological information in accordance with the received temperature information; and causing output, via the input-output device system, of the modified electrophysiological information.

Combinations and sub-combinations of the methods described above may form other systems according to various embodiments.

It should be noted that various embodiments of the present invention include variations of the methods or processes summarized above or otherwise described herein (which should be deemed to include the figures) and, accordingly, are not limited to the actions described or shown in the figures or their ordering, and not all actions shown or described are required according to various embodiments. According to various embodiments, such methods may include more or fewer actions and different orderings of actions. Any of the features of all or part of any one or more of the methods or processes summarized above or otherwise described herein may be combined with any of the other features of all or part of any one or more of the methods or processes summarized above or otherwise described herein.

In addition, a computer program product may be provided that includes program code portions for performing some or all of any one or more of the methods or processes and associated features thereof described herein, when the computer program product is executed by a computer or other computing device or device system. Such a computer program product may be stored on one or more computer-readable storage mediums, also referred to as one or more computer-readable data storage mediums or a computer-readable storage medium system.

In some embodiments, one or more computer-readable storage mediums store a program executable by a data processing device system communicatively connected to an input-output device system. The program may include first reception instructions configured to cause reception of electrophysiological information indicating sensed electrophysiological activity produced within a patient body, the electrophysiological activity sensed via at least a first transducer; second reception instructions configured to cause reception, via the input-output device system, of temperature information indicating temperature at least proximate the first transducer; modification instructions configured to cause modification of the received electrophysiological information in accordance with the received temperature information; and output instructions configured to cause output, via the input-output device system, of the modified electrophysiological information. In some embodiments, the one or more computer-readable storage mediums are or consist of one or more non-transitory computer-readable storage mediums.

In some embodiments, one or more computer-readable storage mediums store a program executable by a data processing device system communicatively connected to an input-output device system. The program may include first acquisition instructions configured to cause acquisition of electrophysiological information indicating sensed electrophysiological activity produced in a patient body, the electrophysiological activity sensed via at least a first transducer; second acquisition instructions configured to cause acquisition of temperature information indicating temperature change at least proximate the first transducer; modification instructions configured to cause modification of the electrophysiological information in accordance with the acquired temperature information; and output instructions configured to cause output, via the input-output device system, of the modified electrophysiological information. In some embodiments, the one or more computer-readable storage mediums are or consist of one or more non-transitory computer-readable storage mediums.

In some embodiments, each of any of one or more of the computer-readable data storage medium systems (also referred to as processor-accessible memory device systems) described herein is a non-transitory computer-readable (or processor-accessible) data storage medium system (or memory device system) including or consisting of one or more non-transitory computer-readable (or processor-accessible) storage mediums (or memory devices) storing the respective program(s) which may configure a data processing device system to execute some or all of any of one or more of the methods or processes described herein.

Further, any of all or part of one or more of the methods or processes and associated features thereof discussed herein may be implemented or executed on or by all or part of a device system, apparatus, or machine, such as all or a part of any of one or more of the systems, apparatuses, or machines described herein or a combination or sub-combination thereof.

### BRIEF DESCRIPTION OF THE DRAWINGS

It is to be understood that the attached drawings are for purposes of illustrating aspects of various embodiments and may include elements that are not to scale.

FIG. 1 includes a schematic representation of an electrophysiological information processing system or a controller system thereof according to various example embodiments, the electrophysiological information processing system including a data processing device system, an input-output device system, and a memory device system.

FIG. 2 includes a cutaway diagram of a heart showing a structure of a transducer-based device percutaneously placed in a left atrium of a heart, according to various example embodiments, the transducer-based device configured to detect electrophysiological information.

FIG. 3A includes a partially schematic representation of an electrophysiological information processing system, according to various example embodiments, the electrophysiological information processing system representing at least a particular implementation of the electrophysiological information processing system of FIG. 1, including a structure of a transducer-based device shown in a delivery or unexpanded configuration, according to some embodiments, the transducer-based device configured to detect electrophysiological information.

FIG. 3B includes a representation of the structure of the transducer-based device configured to detect electrophysiological information of FIG. 3A in a deployed or expanded configuration, according to some embodiments.

FIG. 4 includes a representation of a transducer-based device that includes a flexible circuit structure, according to some embodiments, the transducer-based device configured to detect electrophysiological information.

FIGS. 5A and 5B illustrate electrograms recorded by a transducer-based device submerged in a sodium bicarbonate solution as a test to illustrate temperature change influence on electrograms, where the electrogram of FIG. 5A was recorded in room temperature solution, and the electrogram of FIG. 5B was recorded in the same solution, but with injected warmer solution to cause a temperature change during the respective electrogram detection.

FIG. 6 illustrates changes in an electrogram and temperature caused by a cessation of delivery of radiofrequency (“RF”) energy.

FIG. 7 illustrates programmed configurations of a data processing device system or methods executed by a data processing device system to process electrophysiological information, according to some embodiments.

FIG. 8 illustrates measured electrophysiological voltage data, measured temperature data, and a resulting modification of the electrophysiological voltage data produced by reducing temperature-change-based noise in the measured electrophysiological voltage data, according to some embodiments of the present invention.

### DETAILED DESCRIPTION

At least some embodiments of the present invention improve upon safety, efficiency, and effectiveness of electrophysiological information processing systems. In some embodiments, undesired time-varying noise in sensed electrophysiological signals is at least managed or reduced. In some embodiments, undesired temperature induced noise in sensed electrophysiological signals is at least managed or reduced. It should be noted, however, that the invention is not limited to these, or any other embodiments or examples provided herein, which are referred to for purposes of illustration only. In this regard, for example, while addressing potential undesired time-varying noise effects or undesired temperature-induced noise effects may provide benefits in some embodiments of the present invention, such embodiments may have other benefits, and other embodiments may also have at least some of the same or different benefits.

In this regard, in the descriptions herein, certain specific details are set forth in order to provide a thorough understanding of various embodiments of the invention. However, one skilled in the art will understand that the invention may be practiced at a more general level without one or more of these details. In other instances, well known structures have not been shown or described in detail to avoid unnecessarily obscuring descriptions of various embodiments of the invention.

Any reference throughout this specification to “one embodiment”, “an embodiment”, “an example embodiment”, “an illustrated embodiment”, “a particular embodiment”, and the like means that a particular feature, structure, or characteristic described in connection with the embodiment is included in at least one embodiment. Thus, any appearance of the phrase “in one embodiment”, “in an embodiment”, “in an example embodiment”, “in this illustrated embodiment”, “in this particular embodiment”, or the like in this specification is not necessarily always referring to one embodiment or a same embodiment. Furthermore, the particular features, structures or characteristics of different embodiments may be combined in any suitable manner to form one or more other embodiments.

Unless otherwise explicitly noted or required by context, the word “or” is used in this disclosure in a non-exclusive sense. In addition, unless otherwise explicitly noted or required by context, the word “set” is intended to mean one or more. For example, the phrase, “a set of objects” means one or more of the objects. In some embodiments, the word “subset” is intended to mean a set having the same or fewer elements of those present in the subset's parent or superset. In other embodiments, the word “subset” is intended to mean a set having fewer elements of those present in the subset's parent or superset. In this regard, when the word “subset” is used, some embodiments of the present invention utilize the meaning that “subset” has the same or fewer elements of those present in the subset's parent or superset, and other embodiments of the present invention utilize the meaning that “subset” has fewer elements of those present in the subset's parent or superset.

Further, the phrase “at least” is or may be used herein at times merely to emphasize the possibility that other elements may exist besides those explicitly listed. However, unless otherwise explicitly noted (such as by the use of the term “only”) or required by context, non-usage herein of the phrase “at least” nonetheless includes the possibility that other elements may exist besides those explicitly listed. For example, the phrase, ‘based at least on A’ includes A as well as the possibility of one or more other additional elements besides A. In the same manner, the phrase, ‘based on A’ includes A, as well as the possibility of one or more other additional elements besides A. However, the phrase, ‘based only on A’ includes only A. Similarly, the phrase ‘configured at least to A’ includes a configuration to perform A, as well as the possibility of one or more other additional actions besides A. In the same manner, the phrase ‘configured to A’ includes a configuration to perform A, as well as the possibility of one or more other additional actions besides A. However, the phrase, ‘configured only to A’ means a configuration to perform only A.

The word “device”, the word “machine”, the word “system”, and the phrase “device system” all are intended to include one or more physical devices or sub-devices (e.g., pieces of equipment) that interact to perform one or more functions, regardless of whether such devices or sub-devices are located within a same housing or different housings. However, it may be explicitly specified according to various embodiments that a device or machine or device system resides entirely within a same housing to exclude embodiments where the respective device, machine, system, or device system resides across different housings. The word “device” may equivalently be referred to as a “device system” in some embodiments.

Further, the phrase “in response to” may be used in this disclosure. For example, this phrase may be used in the following context, where an event A occurs in response to the occurrence of an event B. In this regard, such phrase includes, for example, that at least the occurrence of the event B causes or triggers the event A.

The phrase “pulsed field ablation” (“PFA”) as used in this disclosure refers to an ablation method which employs high voltage pulse delivery in a unipolar or bipolar fashion in proximity to target tissue. Each high voltage pulse can be a monophasic pulse including a single polarity, or a biphasic pulse including a first component having a first particular polarity and a second component having a second particular polarity opposite the first polarity. In some embodiments, the second component of the biphasic pulse follows immediately after the first component of the biphasic pulse. In some embodiments, the first and second components of the biphasic pulse are temporally separated by a relatively small time interval. The electric field applied by the high voltage pulses physiologically changes the tissue cells to which the energy is applied (e.g., puncturing or perforating the cell membrane to form various pores therein). If a lower field strength is established, the formed pores may close in time and cause the cells to maintain viability (e.g., a process sometimes referred to as reversible electroporation). If the field strength that is established is greater, then permanent, and sometimes larger, pores form in the tissue cells, the pores allowing leakage of cell contents, eventually resulting in cell death (e.g., a process sometimes referred to as irreversible electroporation).

The word “fluid” as used in this disclosure should be understood to include any fluid that can be contained within a bodily cavity or can flow into or out of, or both into and out of a bodily cavity via one or more bodily openings positioned in fluid communication with the bodily cavity. In the case of cardiac applications, fluid such as blood will flow into and out of various intracardiac cavities (e.g., a left atrium or a right atrium). In some embodiments, the fluid is not a bodily fluid, but rather a fluid (e.g., saline, dyes, medicants) that is introduced into the body according to various diagnostic or therapeutic procedures.

The phrase “bodily opening” as used in this disclosure should be understood to include a naturally occurring bodily opening or channel or lumen; a bodily opening or channel or lumen formed by an instrument or tool using techniques that can include, but are not limited to, mechanical, thermal, electrical, chemical, and exposure or illumination techniques; a bodily opening or channel or lumen formed by trauma to a body; or various combinations of one or more of the above. Various elements having respective openings, lumens or channels and positioned within the bodily opening (e.g., a catheter sheath) may be present in various embodiments. These elements may provide a passageway through a bodily opening for various devices employed in various embodiments.

The words “bodily cavity” as used in this disclosure should be understood to mean a cavity in a body. The bodily cavity may be a cavity or chamber provided in a bodily organ (e.g., an intracardiac cavity of a heart). The bodily cavity may be formed by an instrument or tool using techniques that can include, but are not limited to, mechanical, thermal, electrical, chemical, and exposure or illumination techniques.

The word “tissue” as used in some embodiments in this disclosure should be understood to include any surface-forming tissue that is used to form a surface of a body or a surface within a bodily cavity, a surface of an anatomical feature or a surface of a feature associated with a bodily opening positioned in fluid communication with the bodily cavity. The tissue can include part, or all, of a tissue wall or membrane that defines a surface of the bodily cavity. In this regard, the tissue can form an interior surface of the cavity that surrounds a fluid within the cavity. In the case of cardiac applications, tissue can include tissue used to form an interior surface of an intracardiac cavity such as a left atrium or a right atrium. In some embodiments, the word tissue can refer to a tissue having fluidic properties (e.g., blood) and may be referred to as fluidic tissue.

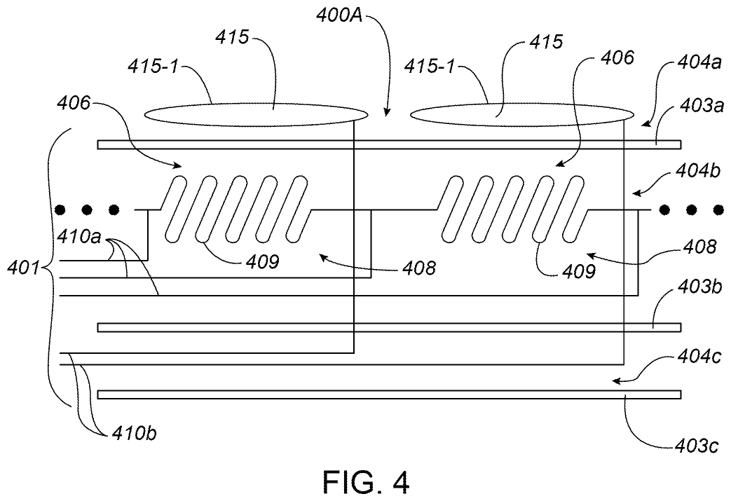

The term “transducer” as used in this disclosure should be interpreted broadly as any device capable of transmitting or delivering energy, distinguishing between fluid and tissue, sensing temperature, creating heat, ablating tissue, sensing, sampling or measuring electrical activity of a tissue surface (e.g., sensing, sampling or measuring intracardiac electrograms, or sensing, sampling or measuring intracardiac voltage data), stimulating tissue, or any combination thereof. A transducer may convert input energy of one form into output energy of another form. Without limitation, a transducer may include an electrode that functions as, or as part of, a sensing device included in the transducer, an energy delivery device included in the transducer, or both a sensing device and an energy delivery device included in the transducer. A transducer may be constructed from several parts, which may be discrete components or may be integrally formed. In this regard, although transducers, electrodes, or both transducers and electrodes are referenced with respect to various embodiments, it is understood that other transducers or transducer elements may be employed in other embodiments. It is understood that a reference to a particular transducer in various embodiments may also imply a reference to an electrode, as an electrode may be part of the transducer as shown, e.g., with FIG. 4 discussed below.

The term “activation” as used in this disclosure should be interpreted broadly as making active a particular function as related to various transducers disclosed in this disclosure, according to some embodiments. Particular functions may include, but are not limited to, tissue ablation (e.g., thermal ablation, cryoablation, PFA), sensing, sampling or measuring electrophysiological activity (e.g., sensing, sampling or measuring electrogram or electrocardiogram information or sensing, sampling or measuring intracardiac voltage data), sensing, sampling or measuring temperature and sensing, sampling or measuring electrical characteristics (e.g., tissue impedance or tissue conductivity). For example, in some embodiments, activation of a tissue ablation function of a particular transducer is initiated by causing energy sufficient for tissue ablation from an energy source device system to be delivered to the particular transducer. Also, in this example, the activation can last for a duration of time concluding when the ablation function is no longer active, such as when energy sufficient for the tissue ablation is no longer provided to the particular transducer. In some contexts, however, the word “activation” can merely refer to the initiation of the activating of a particular function, as opposed to referring to both the initiation of the activating of the particular function and the subsequent duration in which the particular function is active. In these contexts, the phrase or a phrase similar to “activation initiation” may be used.

The word electrophysiology and derivatives thereof pertain to the biomedical field dealing with the study of electric activity in the body. Electrophysiology includes the study of the production of electrical activity and the effects of that electrical activity on the body.

In the following description, some embodiments of the present invention may be implemented at least in part by a data processing device system or a controller system configured by a software program. Such a program may equivalently be implemented as multiple programs, and some, or all, of such software program(s) may be equivalently constructed in hardware. In this regard, reference to “a program” should be interpreted to include one or more programs.

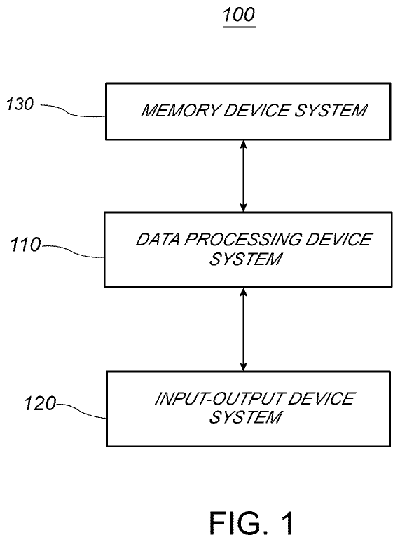

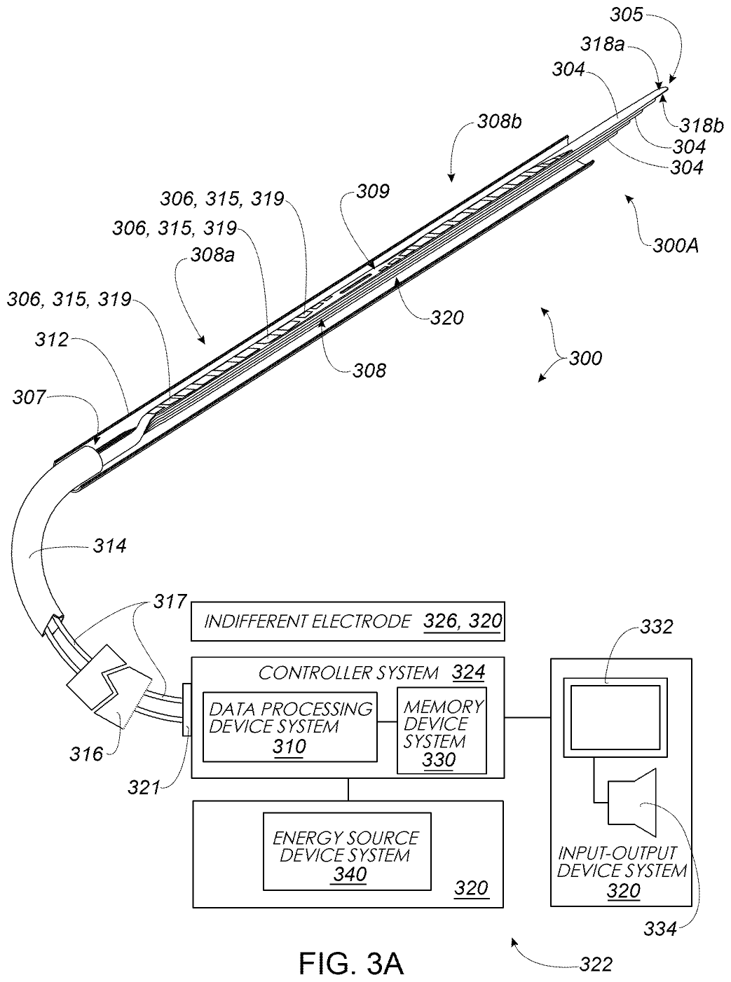

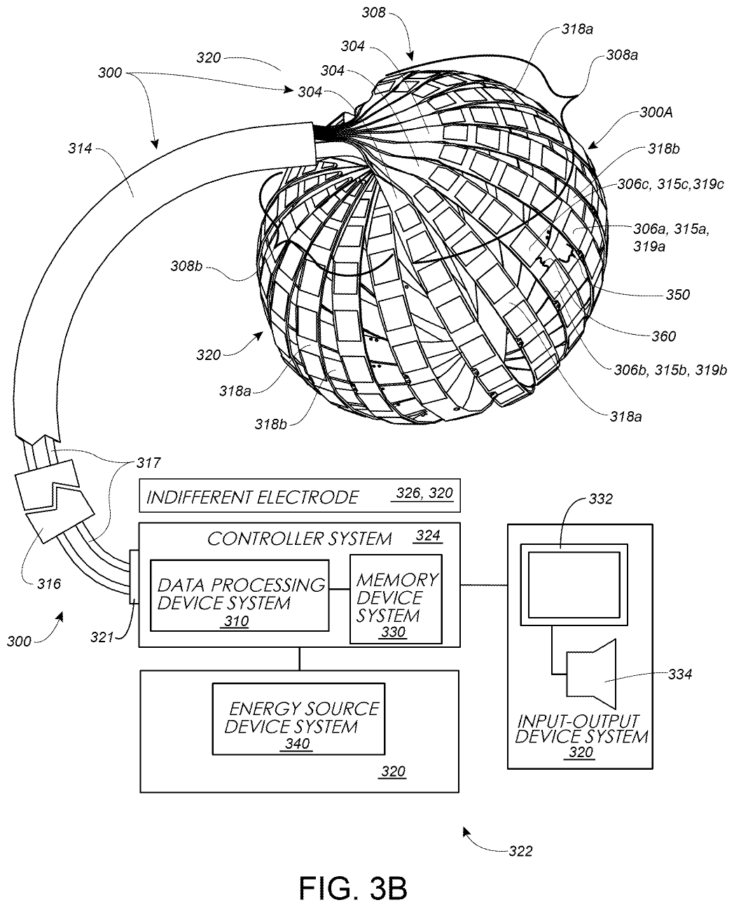

The term “program” in this disclosure should be interpreted as a set of instructions or modules that can be executed by one or more components in a system, such as a controller system or a data processing device system, in order to cause the system to perform one or more operations. The set of instructions or modules may be stored by any kind of memory device, such as those described subsequently with respect to the memory device system 130 or 330 shown in FIGS. 1 and 3, respectively. In addition, this disclosure sometimes describes that the instructions or modules of a program are configured to cause the performance of a function. The phrase “configured to” in this context is intended to include at least (a) instructions or modules that are presently in a form executable by one or more data processing devices to cause performance of the function (e.g., in the case where the instructions or modules are in a compiled and unencrypted form ready for execution), and (b) instructions or modules that are presently in a form not executable by the one or more data processing devices, but could be translated into the form executable by the one or more data processing devices to cause performance of the function (e.g., in the case where the instructions or modules are encrypted in a non-executable manner, but through performance of a decryption process, would be translated into a form ready for execution). The word “module” can be defined as a set of instructions. In some instances, this disclosure describes that the instructions or modules of a program perform a function. Such descriptions should be deemed to be equivalent to describing that the instructions or modules are configured to cause the performance of the function.

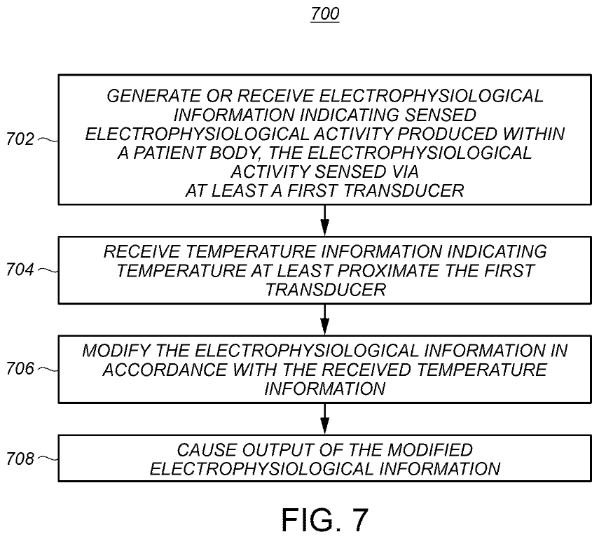

Example methods are described herein with respect to FIG. 7, which includes blocks associated with actions, computer-executable instructions, or both, according to various embodiments. It should be noted that the respective instructions associated with any such blocks therein need not be separate instructions and may be combined with other instructions to form a combined instruction set. The same set of instructions may be associated with more than one block. In this regard, the block arrangement shown in FIG. 7 is not limited to an actual structure of any program or set of instructions or required ordering of method tasks, and such FIG. 7, according to some embodiments, merely illustrates the tasks that instructions are configured to perform, for example, upon execution by a data processing device system, e.g., in conjunction with interactions with one or more other devices or device systems.

Each of the phrases “derived from” or “derivation of” or “derivation thereof” or the like may be used herein to mean to come from at least some part of a source, be created from at least some part of a source, or be developed as a result of a process in which at least some part of a source forms an input. For example, a data set derived from some particular portion of data may include at least some part of the particular portion of data, or may be created from at least part of the particular portion of data, or may be developed in response to a data manipulation process in which at least part of the particular portion of data forms an input. In some embodiments, a data set may be derived from a subset of the particular portion of data. In some embodiments, the particular portion of data is analyzed to identify a particular subset of the particular portion of data, and a data set is derived from the subset. In various ones of these embodiments, the subset may include some, but not all, of the particular portion of data. In some embodiments, changes in least one part of a particular portion of data may result in changes in a data set derived at least in part from the particular portion of data.

In this regard, each of the phrases “derived from” or “derivation of” or “derivation thereof” or the like may be used herein merely to emphasize the possibility that such data or information may be modified or subject to one or more operations. For example, if a device generates first data for display, the process of converting the generated first data into a format capable of being displayed may alter the first data. This altered form of the first data may be considered a derivative or derivation of the first data. For instance, the first data may be a one-dimensional array of numbers, but the display of the first data may be a color-coded bar chart representing the numbers in the array. For another example, if the above-mentioned first data is transmitted over a network, the process of converting the first data into a format acceptable for network transmission or understanding by a receiving device may alter the first data. As before, this altered form of the first data may be considered a derivative or derivation of the first data. For yet another example, generated first data may undergo a mathematical operation, a scaling, or a combining with other data to generate other data that may be considered derived from the first data. In this regard, it can be seen that data is commonly changing in form or being combined with other data throughout its movement through one or more data processing device systems, and any reference to information or data herein is intended in some embodiments to include these and like changes, regardless of whether or not the phrase “derived from” or “derivation of” or “derivation thereof” or the like is used in reference to the information or data. As indicated above, usage of the phrase “derived from” or “derivation of” or “derivation thereof” or the like merely emphasizes the possibility of such changes. Accordingly, in some embodiments, the addition of or deletion of the phrase “derived from” or “derivation of” or “derivation thereof” or the like should have no impact on the interpretation of the respective data or information. For example, the above-discussed color-coded bar chart may be considered a derivative of the respective first data or may be considered the respective first data itself.

In some embodiments, the term “adjacent”, the term “proximate”, and the like refer at least to a sufficient closeness between the objects or events defined as adjacent, proximate, or the like, to allow the objects or events to interact in a designated way. For example, in the case of physical objects, if object A performs an action on an adjacent or proximate object B, objects A and B would have at least a sufficient closeness to allow object A to perform the action on object B. In this regard, some actions may require contact between the associated objects, such that if object A performs such an action on an adjacent or proximate object B, objects A and B would be in contact, for example, in some instances or embodiments where object A needs to be in contact with object B to successfully perform the action. In some embodiments, the term “adjacent”, the term “proximate”, and the like additionally or alternatively refer to objects or events that do not have another substantially similar object or event between them. For example, object or event A and object or event B could be considered adjacent or proximate (e.g., physically or temporally) if they are immediately next to each other (with no other object or event between them) or are not immediately next to each other but no other object or event that is substantially similar to object or event A, object or event B, or both objects or events A and B, depending on the embodiment, is between them. In some embodiments, the term “adjacent”, the term “proximate”, and the like additionally or alternatively refer to at least a sufficient closeness between the objects or events defined as adjacent, proximate, and the like, the sufficient closeness being within a range that does not place any one or more of the objects or events into a different or dissimilar region or time period, or does not change an intended function of any one or more of the objects or events or of an encompassing object or event that includes a set of the objects or events. Different embodiments of the present invention adopt different ones or combinations of the above definitions. Of course, however, the term “adjacent”, the term “proximate”, and the like are not limited to any of the above example definitions, according to some embodiments. In addition, the term “adjacent” and the term “proximate” do not have the same definition, according to some embodiments.

FIG. 1 schematically illustrates a portion of an electrophysiological information processing system or controller system thereof 100 that may be employed to at least to measure electrophysiological information. The system 100 includes a data processing device system 110, an input-output device system 120, and a processor-accessible memory device system 130. The processor-accessible memory device system 130 and the input-output device system 120 are communicatively connected to the data processing device system 110. According to some embodiments, various components such as data processing device system 110, input-output device system 120, and processor-accessible memory device system 130 form at least part of a controller system (e.g., controller system 324 shown in FIG. 3).

The data processing device system 110 includes one or more data processing devices that implement or execute, in conjunction with other devices, such as those in the system 100, various methods and functions described herein, including those described with respect to methods exemplified in FIG. 7. Each of the phrases “data processing device”, “data processor”, “processor”, “controller”, “computing device”, “computer” and the like is intended to include any data or information processing device, such as a central processing unit (CPU), a control circuit, a desktop computer, a laptop computer, a mainframe computer, a tablet computer, a personal digital assistant, a cellular or smart phone, and any other device for processing data, managing data, or handling data, whether implemented with electrical, magnetic, optical, or biological components, or otherwise.

The memory device system 130 includes one or more processor-accessible memory devices configured to store one or more programs and information, including the program(s) and information needed to execute the methods or functions described herein, including those described with respect to method FIG. 7. The memory device system 130 may be a distributed processor-accessible memory device system including multiple processor-accessible memory devices communicatively connected to the data processing device system 110 via a plurality of computers and/or devices. However, the memory device system 130 need not be a distributed processor-accessible memory system and, consequently, may include one or more processor-accessible memory devices located within a single data processing device or housing.

Each of the phrases “processor-accessible memory” and “processor-accessible memory device” and the like is intended to include any processor-accessible data storage device or medium, whether volatile or nonvolatile, electronic, magnetic, optical, or otherwise, including but not limited to, registers, hard disk drives, Compact Discs, DVDs, flash memories, ROMs, and RAMs. In some embodiments, each of the phrases “processor-accessible memory” and “processor-accessible memory device” is intended to include or be a processor-accessible (or computer-readable) data storage medium. In some embodiments, each of the phrases “processor-accessible memory” and “processor-accessible memory device” is intended to include or be a non-transitory processor-accessible (or computer-readable) data storage medium. In some embodiments, the processor-accessible memory device system 130 may be considered to include or be a non-transitory processor-accessible (or computer-readable) data storage medium system. And, in some embodiments, the memory device system 130 may be considered to include or be a non-transitory processor-accessible (or computer-readable) storage medium system or data storage medium system including or consisting of one or more non-transitory processor-accessible (or computer-readable) storage or data storage mediums.

The phrase “communicatively connected” is intended to include any type of connection, whether wired or wireless, between devices, data processors, or programs between which data may be communicated. Further, the phrase “communicatively connected” is intended to include a connection between devices or programs within a single data processor or computer, a connection between devices or programs located in different data processors or computers, and a connection between devices not located in data processors or computers at all. In this regard, although the memory device system 130 is shown separately from the data processing device system 110 and the input-output device system 120, one skilled in the art will appreciate that the memory device system 130 may be located completely or partially within the data processing device system 110 or the input-output device system 120. Further in this regard, although the input-output device system 120 is shown separately from the data processing device system 110 and the memory device system 130, one skilled in the art will appreciate that such system may be located completely or partially within the data processing system 110 or the memory device system 130, for example, depending upon the contents of the input-output device system 120. Further still, the data processing device system 110, the input-output device system 120, and the memory device system 130 may be located entirely within the same device or housing or may be separately located, but communicatively connected, among different devices or housings. In the case where the data processing device system 110, the input-output device system 120, and the memory device system 130 are located within the same device, the system 100 of FIG. 1 can be implemented by a single application-specific integrated circuit (ASIC) in some embodiments.

The input-output device system 120 may include a mouse, a keyboard, a touch screen, another computer, or any device or combination of devices from which a desired selection, desired information, instructions, or any other data is input to the data processing device system 110. The input-output device system 120 may include a user-activatable control system that is responsive to a user action. The user-activatable control system may include at least one control element that may be activated or deactivated on the basis of a particular user action. The input-output device system 120 may include any suitable interface for receiving information, instructions, or any data from other devices and systems described in various ones of the embodiments. In this regard, the input-output device system 120 may include various ones of other systems described in various embodiments. For example, the input-output device system 120 may include at least a portion of a transducer-based device system. The phrase “transducer-based device system” is intended to include one or more physical systems that include various transducers. The phrase “transducer-based device” is intended to include one or more physical devices that include various transducers. A catheter device system that includes one or more transducers or an implant device system that includes one or more transducers may be considered a transducer-based device or device system, according to some embodiments.

The input-output device system 120 also may include an image generating device system, a display device system, a speaker or audio output device system, a computer, a processor-accessible memory device system, a network-interface card or network-interface circuitry, or any device or combination of devices to which information, instructions, or any other data is output by the data processing device system 110. In this regard, the input-output device system 120 may include various other devices or systems described in various embodiments. The input-output device system 120 may include any suitable interface for outputting information, instructions, or data to other devices and systems described in various ones of the embodiments. If the input-output device system 120 includes a processor-accessible memory device, such memory device may, or may not, form part, or all, of the memory device system 130. The input-output device system 120 may include any suitable interface for outputting information, instructions, or data to other devices and systems described in various ones of the embodiments. In this regard, the input-output device system 120 may include various other devices or systems described in various embodiments.

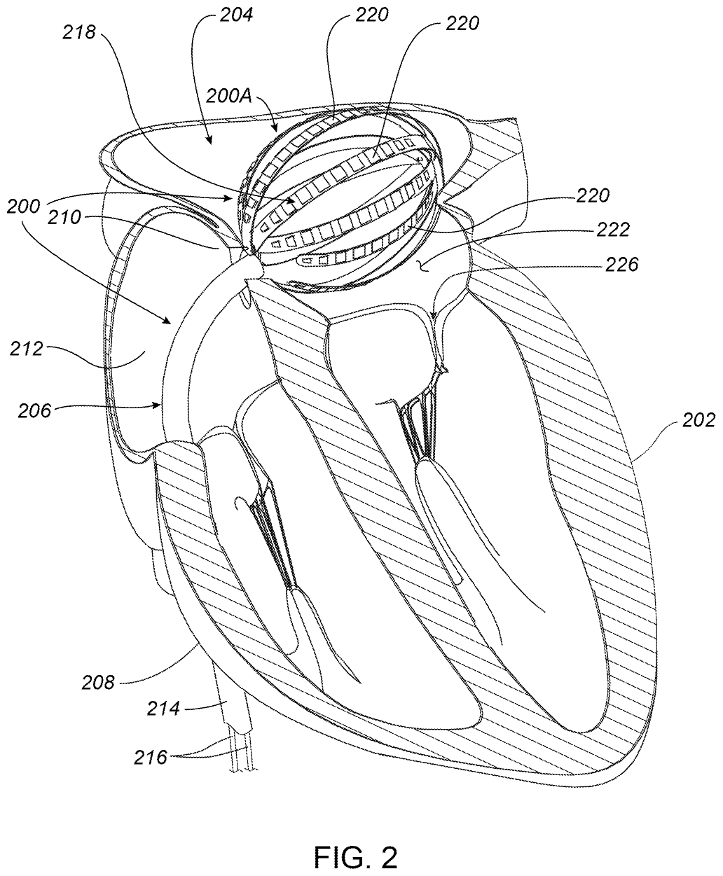

According to some embodiments of the present invention, the system 100 includes some, or all, of the system 200 shown in FIG. 2, or vice versa. In some embodiments, the system 100 includes some, or all, of the system 300 in FIG. 3, or vice versa. In this regard, the system 200, the system 300, or each of the system 200 and the system 300 may be a particular implementation of the system 100, according to some embodiments. Each of at least part of the transducer-based device system 400A in FIG. 4 may be part of the system 100, the system 200, or the system 300, according to various embodiments.

Various embodiments of transducer-based devices are described herein. Some of the described devices are transducer-based devices (e.g., catheter devices) that are percutaneously or intravascularly deployed. Some of the described devices are movable between a delivery or unexpanded configuration (e.g., FIG. 3A discussed below) in which a portion of the device is sized for passage through a bodily opening leading to a bodily cavity, and an expanded or deployed configuration (e.g., FIG. 3B discussed below) in which the portion of the device has a size too large for passage through the bodily opening leading to the bodily cavity. An example of an expanded or deployed configuration, in some embodiments, is when the portion of the transducer-based device is in its intended-deployed-operational state, which may be inside the bodily cavity when, e.g., performing a therapeutic or diagnostic procedure for a patient, or which may be outside the bodily cavity when, e.g., performing testing, quality control, or other evaluation of the device. Another example of the expanded or deployed configuration, in some embodiments, is when the portion of the transducer-based device is being changed from the delivery configuration to the intended-deployed-operational state to a point where the portion of the device now has a size too large for passage through the bodily opening leading to the bodily cavity. According to some embodiments, various ones of the transducer-based devices are configured to detect or sense electrophysiological activity while at least a portion thereof (e.g., a sensing portion such as an electrode) is in contact with a tissue surface. For instance, in some embodiments, a level of contact between at least a part of a transducer-based device and a tissue surface of a bodily cavity may be determined by one or more temperature sensors, such as temperature sensors 408 discussed in more detail below with respect to FIG. 4, which may be located in transducers and can detect convective cooling effects caused by blood flow. Such convective cooling effects typically are proportional to a level of tissue contact in some embodiments. For example, a temperature sensor of a first transducer fully in contact with tissue will experience less convective cooling effects than a temperature sensor of a second transducer not at all in contact with tissue due to the second transducer's greater exposure to blood flow by not being in tissue contact. However, other mechanisms for determining a level of tissue contact may be utilized in some embodiments. See, e.g., U.S. Pat. No. 8,906,011, issued Dec. 9, 2014 (“Gelbart et al.”). In some embodiments, various ones of the transducer-based devices are configured to not contact a tissue surface while detecting or sensing electrophysiological activity. In some embodiments, various electrophysiological sensing portions of an employed transducer-based device are configured to sense electrophysiological information while the various electrophysiological sensing portions do not contact a tissue surface.

Although various transducer-based devices described in this disclosure may be represented as catheter-based devices, according to some embodiments, other embodiments may employ other forms of transducer-based devices. For example, in some embodiments, the transducer-based device configured to sense electrophysiological activity may take the form of an implantable device. Cardiac implantable electronic devices (CIEDs) including pacemakers, implantable cardioverter defibrillators (ICDs), cardiac resynchronization therapy (CRT) devices, and implanted rhythm monitors typically have remote monitoring (RM) of device function and patient health characteristics such as electrophysiological information. In some embodiments, a transducer-based device configured to sense electrophysiological activity may take the form of a device that is applied to an external surface of the body. Electrocardiograms (ECG/EKG) are an example of measured electrophysiological activity sensed by “external” transducer-based devices.

In some example embodiments, various employed transducer-based devices include transducers that sense characteristics (e.g., convective cooling, permittivity, force) that distinguish between fluid, such as a fluidic tissue (e.g., blood), and tissue forming an interior surface of the bodily cavity. Such sensed characteristics may be utilized, e.g., by a data processing device system (e.g., 110, 324) to determine whether a transducer that sensed such characteristics is in contact with a tissue surface of the bodily cavity. Such sensed characteristics can also allow a medical system to map the cavity, for example, using positions of openings or ports into and out of the cavity to determine a position or orientation (e.g., pose), or both, of the portion of the device in the bodily cavity. In some example embodiments, the described systems employ a navigation system or electro-anatomical mapping system including electromagnetic-based systems and electropotential-based systems to determine a positioning of a portion of a device in a bodily cavity. In some example embodiments, the described devices are capable of ablating tissue in a desired pattern within the bodily cavity (for example using thermal ablation or PFA techniques). In some example embodiments, the devices are capable of providing stimulation (e.g., electrical stimulation) to tissue within the bodily cavity. Electrical stimulation may include pacing.

FIG. 2 is a representation of a transducer-based device system 200 including a device 200A useful in treating a bodily organ, for example, a heart 202, according to one example embodiment. Such transducer-based device system 200 may form at least part of an electrophysiological information processing system, according to some embodiments. Device 200A can be percutaneously or intravascularly inserted into a portion of the heart 202, such as an intracardiac cavity like left atrium 204. In this example, the device 200A is part of a catheter 206 inserted via the inferior vena cava 208 and penetrating through a bodily opening in transatrial septum 210 from right atrium 212. In other embodiments, other paths may be taken.

Catheter 206 includes an elongated flexible rod or shaft member 214 appropriately sized to be delivered percutaneously or intravascularly. Various portions of catheter 206 may be steerable. Catheter 206 may include one or more lumens. The lumen(s) may carry one or more communications or power paths, or both. For example, the lumens(s) may carry one or more electrical conductors 216 (two shown in some embodiments although more may be present in other embodiments). Electrical conductors 216 provide electrical connections to system 200 that are accessible (e.g., by a controller system or data processing device system) externally from a patient in which the device 200A is inserted.

Device 200A includes a frame or structure 218 which assumes an unexpanded configuration for delivery to left atrium 204, according to some embodiments. In some embodiments, structure 218 is expanded (e.g., shown in a deployed or expanded configuration in FIG. 2) upon delivery to left atrium 204 to position a plurality of transducers 220 (three called out in FIG. 2) proximate the interior surface formed by tissue 222 of left atrium 204. In some embodiments, at least some of the transducers 220 are configured to sense a physical characteristic of a fluid (e.g., blood), or tissue 222, or both, and based on the sensed physical characteristic(s), the at least some of the transducers 220 may be configured to determine a position or orientation (e.g., pose), or both, of a portion of system 200 within, or with respect to left atrium 204. For example, transducers 220 may be configured to determine a location of pulmonary vein ostia (not shown) or a mitral valve 226, or both. In some embodiments, at least some of the transducers 220 may be configured to selectively ablate portions of the tissue 222. In some embodiments, some of the transducers 220 may be configured to ablate a pattern around the bodily openings, ports, or pulmonary vein ostia, for instance, to reduce or eliminate the occurrence of atrial fibrillation. In some embodiments, at least some of the transducers 220 are configured to ablate cardiac tissue, such as by thermal ablation or PFA. In some embodiments, at least some of the transducers 220 are configured to sense or sample electrophysiological information (e.g., intracardiac voltage data or intracardiac electrogram data). In some embodiments, at least some of the transducers 220 are configured to sense or sample electrophysiological information (e.g., intracardiac voltage data or intracardiac electrogram data), while at least some of the transducers 220 are concurrently ablating cardiac tissue. In some embodiments, at least one of the sensing or sampling transducers 220 is provided by at least one of the ablating transducers 220. In some embodiments, at least a first one of the transducers 220 is configured to sense or sample electrophysiological information (e.g., intracardiac voltage data or intracardiac electrogram data) at a location at least proximate to a tissue location ablated by at least a second one of the transducers 220. In some embodiments, the first one of the transducers 220 is other than the second one of the transducers 220. In various embodiments, each of at least some of the transducers 220 includes an electrode. In various embodiments, each of at least some of the transducers 220 includes an electrode configured to deliver ablative energy to tissue.

FIGS. 3A and 3B (collectively, FIG. 3) include a transducer-based device system 300 (e.g., a portion thereof shown schematically) that includes a device 300A, according to one illustrated embodiment. Such transducer-based device system 300 may form at least part of an electrophysiological information processing system, according to some embodiments. Each of FIGS. 3A and 3B may represent one or more implementations of the electrophysiological information processing system 100 of FIG. 1, according to some embodiments. In this regard, the system 300 in each of FIGS. 3A and 3B may be configured to deliver energy to each of one or more elements, such as one or more transducers or one or more electrodes. The device 300A may include at least one hundred electrodes 315, but need not include that many or may include more. FIG. 3A illustrates the device 300A in the delivery or unexpanded configuration, according to various example embodiments, and FIG. 3B illustrates the device 300A in the deployed or expanded configuration, according to some embodiments.

In this regard, the device 300A includes a plurality of elongate members 304 (three called out in each of FIGS. 3A and 3B) and a plurality of transducers 306 (three called out in FIG. 3A and three called out in FIG. 3B as 306a, 306b and 306c). In some embodiments, the transducers 306 have the configuration of the transducers 220 in FIG. 2. In some embodiments, the transducers 306 are formed as part of, or are located on, the elongate members 304. In some embodiments, the elongate members 304 are arranged as a frame or structure 308 that is selectively movable between an unexpanded or delivery configuration (e.g., as shown in FIG. 3A) and an expanded or deployed configuration (e.g., as shown in FIG. 3B) that may be used to position elongate members 304 or various one of the transducers 306 against a tissue surface within the bodily cavity or position the elongate members 304 in the vicinity of, or in contact with, the tissue surface.

Although FIGS. 3A and 3B show a particular number of elongate members 304 with respective particular lengths thereof, some embodiments have more or fewer elongate members 304 with the same or different respective particular lengths thereof. In this regard, varying the number and lengths of elongate members 304 can change the overall size of structure 308 and the number and density of transducers 306. For example, in some applications and embodiments, it may be desirable to have a smaller spherical size of structure 308, so that the structure 308 can more readily fit into alcoves of a bodily cavity, and such a smaller spherical size may be achieved by reducing the number of elongate members 304 and/or shortening their respective particular lengths, according to some embodiments.

In some embodiments, the structure 308 has a size in the unexpanded or delivery configuration suitable for percutaneous delivery through a bodily opening (e.g., via catheter sheath 312, shown in FIG. 3A, but not shown in FIG. 3B for purposes of clarity) to the bodily cavity. In some embodiments, structure 308 has a size in the expanded or deployed configuration too large for percutaneous delivery through a bodily opening (e.g., via catheter sheath 312) to the bodily cavity. The elongate members 304 may form part of a flexible circuit structure (e.g., such as a flexible printed circuit board (PCB)). The elongate members 304 may include a plurality of different material layers, and each of the elongate members 304 may include a plurality of different material layers, according to various embodiments. The structure 308 may include a shape memory material, for instance, Nitinol. The structure 308 may include a metallic material, for instance, stainless steel, or non-metallic material, for instance polyimide, or both a metallic and a non-metallic material by way of non-limiting example. The incorporation of a specific material into structure 308 may be motivated by various factors including the specific requirements of each of the unexpanded or delivery configuration and expanded or deployed configuration, the required position or orientation (e.g., pose) or both of structure 308 in the bodily cavity, or the requirements for successful PFA of a desired pattern.

The plurality of transducers 306 are positionable within a bodily cavity, for example, by positioning of the structure 308. For instance, in some embodiments, the transducers 306 are able to be positioned in a bodily cavity by movement into, within, or into and within the bodily cavity, with or without a change in a configuration of the plurality of transducers 306 (e.g., a change in a configuration of the structure 308 causes a change in a configuration of the transducers 306 in some embodiments). In some embodiments, the plurality of transducers 306 are arrangeable to form a two- or three-dimensional distribution, grid, or array capable of mapping, ablating, or stimulating an inside surface of a bodily cavity or lumen without requiring mechanical scanning. As shown, for example, in FIG. 3A, the plurality of transducers 306 are arranged in a distribution receivable in a bodily cavity (not shown in FIG. 3A). As shown, for example, in FIG. 3A, the plurality of transducers 306 are arranged in a distribution suitable for delivery to a bodily cavity.

FIG. 4 is a schematic side elevation view of at least a portion of a transducer-based device system 400A that includes a flexible circuit structure 401 that is employed to provide a plurality of transducers 406 (two called out) according to an example embodiment. Such transducers may correspond to transducers 220 or 306, according to various embodiments. In some embodiments, the flexible circuit structure 401 may form part of a structure (e.g., structure 308) that is selectively movable between a delivery configuration sized for percutaneous delivery and expanded or deployed configurations sized too large for percutaneous delivery. In some embodiments, the flexible circuit structure 401 may be located on, or form at least part of, a structural component (e.g., elongate member 304) of a transducer-based device system.

The flexible circuit structure 401 may be formed by various techniques including flexible printed circuit techniques. In some embodiments, the flexible circuit structure 401 includes various layers including flexible layers 403a, 403b and 403c (e.g., collectively flexible layers 403). In some embodiments, each of flexible layers 403 includes an electrical insulator material (e.g., polyimide). One or more of the flexible layers 403 can include a different material than another of the flexible layers 403. In some embodiments, the flexible circuit structure 401 includes various electrically conductive layers 404a, 404b and 404c (collectively electrically conductive layers 404) that are interleaved with the flexible layers 403. In some embodiments, each of the electrically conductive layers 404 is patterned to form various electrically conductive elements. For example, in some embodiments, electrically conductive layer 404a is patterned to form a respective electrode 415 of each of the transducers 406. In some embodiments, electrodes 415 correspond to electrodes 315. Electrodes 415 have respective electrode edges 415-1 that form a periphery of an electrically conductive surface associated with the respective electrode 415. It is noted that other electrodes employed in other embodiments may have electrode edges arranged to form different electrode shapes.

Electrically conductive layer 404b is patterned, in some embodiments, to form respective temperature sensors 408 for each of the transducers 406 as well as various leads 410a arranged to provide electrical energy to the temperature sensors 408. In some embodiments, each temperature sensor 408 includes a patterned resistive member 409 (two called out) having a predetermined electrical resistance. In some embodiments, each resistive member 409 includes a metal having relatively high electrical conductivity characteristics (e.g., copper). In some embodiments, electrically conductive layer 404c is patterned to provide portions of various leads 410b arranged to provide an electrical communication path to electrodes 415. In some embodiments, leads 410b are arranged to pass through vias (not shown) in flexible layers 403a and 403b to connect with electrodes 415. Although FIG. 4 shows flexible layer 403c as being a bottom-most layer, some embodiments may include one or more additional layers underneath flexible layer 403c, such as one or more structural layers, such as a steel or composite layer. These one or more structural layers, in some embodiments, are part of the flexible circuit structure 401 and can be part of, e.g., elongate member 304. In some embodiments, the one or more structural layers may include at least one electrically conductive surface (e.g., a metallic surface) exposed to blood flow. In addition, although FIG. 4 shows only three flexible layers 403a-403c and only three electrically conductive layers 404a-404c, it should be noted that other numbers of flexible layers, other numbers of electrically conductive layers, or both, can be included.

In some embodiments, electrodes 415 are employed to selectively deliver thermal ablative energy (e.g., RF ablative energy) to various tissue structures within a bodily cavity (not shown in FIG. 4). In some embodiments, electrodes 415 are employed to selectively deliver PFA high voltage pulses to various tissue structures within a bodily cavity (not shown in FIG. 4) (e.g., an intracardiac cavity or chamber). The PFA high voltage pulses delivered to the tissue structures may be sufficient for ablating portions of the tissue structures. The PFA high voltage pulses delivered to the tissue may be delivered to cause monopolar pulsed field tissue ablation, bipolar pulsed field tissue ablation, or blended monopolar-bipolar pulsed field tissue ablation by way of non-limiting example. The energy that is delivered by each high voltage pulse may be dependent upon factors including the electrode location, size, shape, relationship with respect to another electrode (e.g., the distance between adjacent electrodes that deliver the PFA energy), the presence, or lack thereof, of various material between the electrodes, the degree of electrode-to-tissue contact, and other factors. In some cases, a maximum ablation depth resulting from the delivery of high voltage pulses by a relatively smaller electrode is typically shallower than that of a relatively larger electrode. It is noted that although PFA is considered by some to be a generally non-thermal method for causing cell death, the use of various PFA protocols may cause some degree of thermal damage to tissue of a desired ablation region. For instance, the present inventors recognized that, when relatively high PFA voltages and/or a relatively large number of pulses are employed to ablate the tissue, clinically significant Joule heating of tissue may be encountered during PFA. In some embodiments, each electrode 415 is employed to sense or sample an electrical potential in the tissue proximate the electrode 415 at a same or different time than delivering high voltage output pulses for pulsed field tissue ablation. In some embodiments, each electrode 415 is employed to sense or sample electrophysiological activity in the tissue proximate the electrode 415. In some embodiments, each electrode 415 is employed to sense or sample data in the tissue proximate the electrode 415 from which an electrogram (e.g., an intracardiac electrogram) may be derived. In some embodiments, each resistive member 409 is positioned adjacent a respective one of the electrodes 415. In some embodiments, each of the resistive members 409 is positioned in a stacked or layered array with a respective one of the electrodes 415 to form a respective one of the transducers 406. In some embodiments, the resistive members 409 are connected in series to allow electrical current to pass through all of the resistive members 409. In some embodiments, leads 410a are arranged to allow for a sampling of electrical voltage in between resistive members 409. This arrangement allows for the electrical resistance of each resistive member 409 to be accurately measured. The ability to accurately measure the electrical resistance of each resistive member 409 may be motivated by various reasons including determining temperature values at locations at least proximate the resistive member 409 based at least on changes in the resistance caused by convective cooling effects (e.g., as provided by blood flow). Electrical current driven through each resistive member 409 and voltage across each of the resistive member 409 may be sampled by a sensing system, thereby allowing for the electrical resistance of each of the resistive members 409 to be precisely measured by a controller system (e.g., 324 described below). The resistance of an electrically conductive metal (e.g., copper) changes based on the temperature of the electrically conductive metal. The rate of change is denominated as a temperature coefficient of resistance (“TCR”). According to various embodiments, the resistance of various ones of the resistive members 409 may be related to the temperature of the resistive member 409 by the following relationship shown by equation (1), below:

R=R₀*[1+TCR(T−T₀)], (1)

where:

R is a resistance of the electrically conductive metal at a temperature T;

R₀ is a resistance of the electrically conductive metal at a reference temperature T₀;

TCR is the temperature coefficient of resistance for the reference temperature (i.e., the TCR for copper is 4270 ppm at T₀=0° C.); and

T is the temperature of the electrically conductive metal.

It is noted that other temperatures may be employed according to various embodiments. For example, a temperature sensing transducer may include a thermistor, resistance temperature detector (“RTD”), thermocouple or temperature sensitive diode by way of non-limiting example. In some example embodiments, a transducer (e.g., a resistance temperature detector) can be used to induce a temperature change as well as sensing the temperature change.

In some embodiments, each electrode 415 is employed to sense or sample impedance (or an electrical characteristic related to impedance such as voltage or current) of tissue proximate the electrode 415 at a same or different time than delivering tissue ablative energy. For example, in some embodiments, an impedance sensing system including a voltage sensor, a current sensor, or a combination of a voltage and a current sensor may be provided with the electrode 415 electrically connected to the sensor circuit. In various embodiments, a return electrode (e.g., a second electrode 415 or indifferent electrode 326 (described below)) is electronically coupled to the sensor circuit. In various embodiments, a controller system (e.g., 324 described below) is communicatively connected or coupled to or contains the sensor circuit and is configured to cause an electrical signal set to be applied to the electrode 415 electrode and the return electrode, and to receive a signal set from the sensor circuit in response to the applied signal set. According to various embodiments, the controller system 324 is configured to determine impedance based at least on the received signal set. According to various embodiments, the controller system 324 is configured to determine tissue resistance based at least on the received signal set. According to various embodiments, the controller system 324 is configured to determine a tissue dielectric constant based at least on the received signal set.

Referring again to FIGS. 3A and 3B, according to some embodiments, device 300A communicates with, receives power from, or is controlled by a transducer-activation system 322, which may include a controller system 324 and an energy source device system 340. In some embodiments, the controller system 324 includes a data processing device system 310 and a memory device system 330 that stores data and instructions that are executable by the data processing device system 310 to process information received from other components of the system 300 of FIGS. 3A and 3B or to control operation of components of the system 300 of FIGS. 3A and 3B, for example, by activating various selected transducers 306 to perform tissue ablation or sense tissue characteristics. In this regard, the data processing device system 310 may correspond to at least part of the data processing device system 110 in FIG. 1, according to some embodiments, and the memory device system 330 may correspond to at least part of the memory device system 130 in FIG. 1, according to some embodiments. The energy source device system 340, in some embodiments, is part of an input-output device system 320, which may correspond to at least part of the input-output device system 120 in FIG. 1. The controller system 324 may be implemented by one or more controllers. In some embodiments, the device 300A is considered to be part of the input-output device system 320. The input-output device system 320 may also include a display device system 332, a speaker device system 334, or any other device such as those described above with respect to the input-output device system 120.

In some embodiments, elongate members 304 may form a portion or an extension of control leads 317 that reside, at least in part, in an elongated cable 316 and, at least in part, in a flexible catheter body 314. The control leads terminate at a connector 321 or other interface with the transducer-activation system 322 and provide communication pathways between at least the transducers 306 and the controller 324. The control leads 317 may correspond to electrical conductors 216 in some embodiments.

According to some embodiments, the energy source device system 340 includes various energy source devices (for example a high voltage supply system, in PFA systems). It is noted that, although FIGS. 3A and 3B show a communicative connection between the energy source device system 340 and the controller system 324 (and its data processing device system 310), the energy source device system 340 may also be connected to the transducers 306 via a communicative connection that is independent of the communicative connection with the controller system 324 (and its data processing device system 310). For example, the energy source device system 340 may receive control signals via the communicative connection with the controller system 324 (and its data processing device system 310), and, in response to such control signals, deliver energy to, receive energy from, or both deliver energy to and receive energy from one or more of the transducers 306 via a communicative connection with such transducers 306 (e.g., via one or more communication lines through catheter body 314, elongated cable 316 or catheter sheath 312) that does not pass through the controller system 324. In this regard, the energy source device system 340 may provide results of its delivering energy to, receiving energy from, or both delivering energy to and receiving energy from one or more of the transducers 306 to the controller system 324 (and its data processing device system 310) via the communicative connection between the energy source device system 340 and the controller system 324. In some embodiments, some, or all, of the energy source device system 340 may be considered part of the controller system 324.

The number of energy source devices in the energy source device system 340 may be fewer than the number of transducers in some embodiments. In some embodiments, the energy source device system 340 may, for example, be connected to various selected transducers 306 to selectively provide energy in the form of electrical current or power, light, or low temperature fluid to the various selected transducers 306 to cause ablation of tissue. The energy source device system 340 may, for example, selectively provide energy in the form of electrical current to various selected transducers 306 and measure a temperature characteristic, an electrical characteristic, or both at a respective location at least proximate each of the various transducers 306. The energy source device system 340 may include various electrical current sources or electrical power sources as energy source devices. In some embodiments, an indifferent electrode 326 is provided to receive at least a portion of the energy transmitted by at least some of the transducers 306. Consequently, although not shown in various ones of FIGS. 3, the indifferent electrode 326 may be communicatively connected to the energy source device system 340 via one or more communication lines in some embodiments. In addition, although shown separately in various ones of FIGS. 3, indifferent electrode 326 may be considered part of the energy source device system 340 in some embodiments. In various embodiments, indifferent electrode 326 is positioned on an external surface (e.g., a skin-based surface) of a body that includes the bodily cavity into which at least transducers 306 are to be delivered.

It is understood that input-output device system 320 may include other systems. In some embodiments, input-output device system 320 may optionally include energy source device system 340, device 300A, or both energy source device system 340 and device 300A, by way of non-limiting example. Input-output device system 320 may include the memory device system 330 in some embodiments.

Structure 308 may be delivered and retrieved via a catheter member, for example, a catheter sheath 312. In some embodiments, the structure 308 provides expansion and contraction capabilities for a portion of a medical device (e.g., an arrangement, distribution, or array of transducers 306). The transducers 306 may form part of, be positioned or located on, mounted or otherwise carried on the structure 308, and the structure may be configurable to be appropriately sized to slide within catheter sheath 312 in order to be deployed percutaneously or intravascularly.

FIG. 3A shows one embodiment of such a structure, where the elongate members 304, in some embodiments, are stacked in the delivery or unexpanded configuration to facilitate fitting within the flexible catheter sheath 312. In some embodiments, each of the elongate members 304 includes a respective distal end 305 (only one called out in FIG. 3A), a respective proximal end 307 (only one called out in FIG. 3A) and an intermediate portion 309 (only one called out in FIG. 3A) positioned between the proximal end 307 and the distal end 305. Correspondingly, in some embodiments, structure 308 includes a proximal portion 308a and a distal portion 308b. In some embodiments, the proximal and the distal portions 308a, 308b include respective portions of elongate members 304. The respective intermediate portion 309 of each elongate member 304 may include a first or front surface 318a that is positionable to face an interior tissue surface within a bodily cavity and a second or back surface 318b opposite across a thickness of the intermediate portion 309 from the front surface 318a. In some embodiments, each elongate member 304 includes a twisted portion at a location proximate proximal end 307.

The transducers 306 may be arranged in various distributions or arrangements in various embodiments. In some embodiments, various ones of the transducers 306 are spaced apart from one another in a spaced apart distribution as shown, for example, in at least FIGS. 3A and 3B. In some embodiments, various regions of space are located between various pairs of the transducers 306. For example, in FIG. 3B the system 300 includes at least a first transducer 306a, a second transducer 306b, and a third transducer 306c (all collectively referred to as transducers 306). In some embodiments, each of the first, the second, and the third transducers 306a, 306b and 306c are adjacent transducers in the spaced apart distribution. In some embodiments, the first and the second transducers 306a, 306b are located on different elongate members 304 while the second and the third transducers 306b, 306c are located on a same elongate member 304. In some embodiments, a first region of space 350 is between the first and the second transducers 306a, 306b. In some embodiments, the first region of space 350 is not associated with any physical portion of structure 308. In some embodiments, a second region of space 360 associated with a physical portion of device system 300 (e.g., a portion of an elongate member 304) is between the second and the third transducers 306b, 306c. In some embodiments, each of the first and the second regions of space 350, 360 does not include a transducer or electrode thereof of system 300. In some embodiments, each of the first and the second regions of space 350, 360 does not include any transducer or electrode.

It is noted that other embodiments need not employ a group of elongate members 304 as employed in the illustrated figures. For example, other embodiments may employ a structure including one or more surfaces, at least a portion of the one or more surfaces defining one or more openings in the structure. In these embodiments, a region of space not associated with any physical portion of the structure may extend over at least part of an opening of the one or more openings. In some example embodiments, other structures may be employed to support or carry transducers of a transducer-based device provided by various embodiments described in this disclosure. Basket catheters or balloon catheters may be used to distribute the transducers in a two-dimensional or three-dimensional array. In other example embodiments, other structures may be employed to support or carry transducers of a transducer-based device provided by various flexible circuit structures (e.g., such as the flexible circuit structures associated with FIG. 4, in some embodiments). In some embodiments, an elongated catheter member may be used to distribute the flexible circuit structure-based transducers in a linear or curvilinear array.

In various example embodiments, the energy transmission surface 319 of each electrode 315 is provided by an electrically conductive surface. In some embodiments, each of the electrodes 315 is located on various surfaces of an elongate member 304 (e.g., front surfaces 318a or back surfaces 318b). In some embodiments, various electrodes 315 are located on one, but not both, of the respective front surface 318a and respective back surface 318b of each of various ones of the elongate members 304. For example, various electrodes 315 may be located only on the respective front surfaces 318a of each of the various ones of the elongate members 304. Three of the electrodes 315 are identified as electrodes 315a, 315b, and 315c in FIG. 3B. Three of the energy transmission surfaces 319 are identified as 319a, 319b, and 319c in FIG. 3B. In various embodiments, it is intended or designed to have the entirety of each of various ones of the energy transmission surfaces 319 be available or exposed (e.g., without some obstruction preventing at least some of the ability) to contact non-fluidic tissue at least when structure 308 is positioned in a bodily cavity in the expanded configuration.

In various embodiments, the respective shape of various electrically conductive surfaces (e.g., energy transmission surfaces 319) of various ones of the electrodes 315 vary among the electrodes 315. In various embodiments, one or more dimensions or sizes of various electrically conductive surfaces (e.g., energy transmission surfaces 319) of at least some of the electrodes 315 vary among the electrodes 315. The shape or size variances associated with various ones of the various electrically conductive surfaces of electrodes 315 may be motivated for various reasons. For example, in various embodiments, the shapes or sizes of various ones of the various electrically conductive surfaces of electrodes 315 may be controlled in response to various size or dimensional constraints imposed by structure 308.

It should be noted that the present invention is not limited to any particular transducer-based device transducer arrangement, and the devices 200A, 300A, 400A are provided for illustration purposes only.

Electrophysiological activity generally produces low amplitude signals recorded in the presence of numerous sources of noise and interference. Conventional techniques view this noise as constant or non-time-varying (e.g., “DC”) bias voltage. As a result, common-mode noise rejection or a high-pass filter with a low cut-off frequency (e.g., 0.05 Hz) has been used to remove the DC bias voltage from the measured electrophysiological voltage signals. The present inventors, however, realized that some noise associated with the measured electrophysiological voltage signals may be non-constant or time-varying in nature. For example, the present inventors have determined that temperature-dependent effects may be a source of, or a contributor, of time-varying noise in the measured electrophysiological voltage signals. The present inventors have determined that when temperature changes over time, those temperature-dependent effects are not stable, which in turn, leads to non-DC effects that are not removed with conventional techniques.

Various natural phenomena can result in an electromotive force that is either directly caused by differences in temperature or modulated by them. One example is the Seebeck effect, a physical phenomenon in which electromotive forces are directly caused by temperature gradients. A second example is that of reduction-oxidation reactions, a chemical phenomenon in which a pair of galvanic half-cell reactions produce an electromotive force with the magnitude of that force being a function of temperature.

The Seebeck effect is one of three closely related effects, alongside the Peltier and Thomson effects, collectively referred to as the thermoelectric effect. The Seebeck effect describes a voltage directly produced by a temperature gradient in a conductive material, caused by electrons diffusing in the opposite direction of the thermal gradient (i.e., the thermal gradient directed from hot to cold). Mathematically, the local voltage produced from this effect, E_{emf} may be described by the following equation:

E_{emf}=−SV̅T (2)

where:

S is a material property known as the Seebeck coefficient, and

V̅T is the temperature gradient.

Conversely, in the chemical phenomenon of reduction-oxidation reactions, an electromotive force is induced as a result of a net decrease in Gibbs free energy caused by a pair of half-cell reactions. In these reactions, the change in Gibbs free energy and, therefore, the induced voltage, is a function of temperature. This pair of half-cell reactions may arise due to the use of dissimilar metals in the measuring circuit. For example, cardiac transducer-based devices routinely measure electrophysiological voltage on or within a patient's body, such as inside of the heart using one or more sensing electrodes (e.g., 315, 415). These voltage measurements are often taken with respect to a “neutral pad” or “neutral electrode” (e.g., indifferent electrode 326) placed on the patient's skin to serve as an electrical ground. In some cases, the one or more sensing electrodes (e.g., 315, 415) include a gold composition (e.g., a gold layer over a copper electrode pad) while the neutral pad includes an aluminum composition, thereby generating a bimetallic cell. Alternatively, the half-cell reactions may also arise when the electrodes are formed from a same material, but the electrodes are at different temperatures, thereby generating a thermogalvanic cell. In either case, the induced voltage may be described by the Nernst equation represented as follows:

$${E}_{cell} = {E}_{cell}^{θ} - \frac{RT}{zF} ln {Q}_{r} \qquad ( 3 )$$

where:

T is temperature,

E_{cell} is the resulting voltage,

E_{cell}^{θ} is the voltage under certain standard conditions,

R and F are relevant physical constants,

z is a constant determined by the chemical species involved, and

Qᵣ is a ratio determined by the relative activity or concentration of reagents.

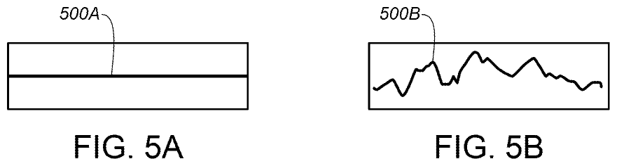

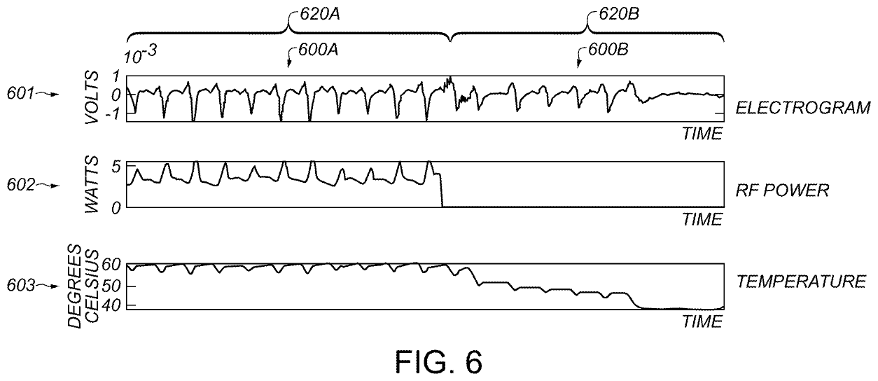

When the temperatures that produce and/or modulate these effects are stable over time, the resulting electrical noise may be considered to produce a constant or DC bias that may be removed or mitigated by conventional techniques. However, when one or more of these temperatures, such as the temperature at the sensing electrode, are time-varying, the resulting electromotive force is also time-varying and may therefore appear unattenuated in the representation of the resulting electrophysiological information (e.g., a cardiac electrogram), degrading the quality thereof. Although the invention is not bound by any particular theory, the inventors hypothesize that changes in the sensed electrophysiological voltages in response to temperature changes, can be described by the Nernst equation (3), according to some embodiments. To provide further support to the hypothesized mechanism of electrical noise caused by temperature variation, the inventors conducted an experiment that was designed to recreate the effect in a controlled environment. In this experiment, a transducer-based device similar to the device of FIG. 3 was immersed in a tank containing a sodium bicarbonate solution at room temperature (approximately 20° C.). Sodium bicarbonate was dissolved in the water thereby creating dissolved ions that allowed the solution to have a conductivity approximating blood. It is noted that a saline solution could also be employed. In the absence of any electrical phenomena representative of electrophysiological activities, electrograms recorded by the transducers (e.g., electrodes) of the transducer-based device are “flat” (for example, as shown in FIG. 5A by electrogram 500A, which was recorded by a particular transducer (e.g., a particular electrode) of the employed transducer-based device). According to the conducted experiment, “temperature variation” was introduced by injecting warm (33° C.) solution onto a subset of the transducers (including the particular transducer) of the immersed transducer-based device. The electrogram 500B in FIG. 5B is the electrogram recorded by the particular transducer during the injection of the warm solution. The significant deviations in electrogram 500B from the “flat” state of electrogram 500A shows the considerable, non-constant noise introduced into the electrogram 500B under the influence of the temperature change. The present inventors note that electrogram deviation would not be readily observed if isothermal solution were to be injected over the transducer. It is noted that electrograms recorded by other transducers of the employed transducer-based device showed similar behavior. The time-varying electrical signal measured in response to a moderate (˜10° C.−13° C.) change in temperature of the surrounding solution demonstrates that temperature dependent phenomena can cause an appreciable change in the recorded electrograms. In addition to demonstrating the hypothesized temperature-dependent noise in a bench-top environment, the present inventors also successfully identified temperature-dependent noise during in-vivo energy delivery. In many cases, the temperature of the transducer portion is closely correlated with delivered energy, especially when ablative energy (e.g., thermal ablative energy and in some cases, PFA energy) is delivered. During ablation, there is often a component of the measured electrophysiological activity that appears to be correlated with variations in the temperature of that electrode, referred to here as temperature-based noise (as opposed to noise imparted onto recorded electrophysiological information from interference effects). FIG. 6 shows three recordings taken during an experiment conducted by the present inventors in which a transducer-based device similar to the device of FIG. 3 was activated, such that a particular transducer (i.e., an electrode in this case) transmitted ablative RF energy for a first period of time 620A, which was immediately succeeded by a second period of time 620B in which no ablative energy was transmitted by the particular transducer. In particular, row 601 in FIG. 6 shows recorded electrophysiological voltages (i.e., in the form of an electrogram in this particular case) sensed by the particular transducer during the first period of time 620A and the second period of time 620B. Row 602 in FIG. 6 shows a graph represented of the RF power delivered by the particular transducer during the first period of time 620A and the lack of transmitted RF power during the second period of time 620B. Row 603 in FIG. 6 shows temperature recorded by the particular transducer during the first period of time 620A and the second period of time 620B. It is noted that rows 601, 602, and 603 are depicted synchronized in time. Observation of the electrogram of row 601 shows a first portion 600A during the first period of time 620B in which RF power is delivered by the particular transducer, and a second portion 600B during the second period of time in which RF power is not delivered by the particular transducer. It is difficult to distinguish any sources of noise acting on the electrogram first portion 600A due to interference caused by the delivery of the RF ablative power during the first period of time 620A. However, the observation of residual temperature (i.e., caused by the ablation process) during the second period of time 620B in FIG. 6 corresponds to the electrogram noise post ablation in the second portion 600B of the electrogram in row 601, after the ablative power delivery has stopped. This provided compelling evidence that described artifacts are mainly related to temperature fluctuation and not merely correlated with it as the result of a common cause (RF energy delivery).

It is noted that, although the present inventors believe that that temperature variation is a large contributing noise factor, the present inventors believe that other contributing factors may also exist. For example, other factors may be associated with, by way of non-limiting example, electromagnetic interference from nearby devices and/or other circuitry within the controller, leakage current from other patient-contacting equipment, far-field noise originating from distant biological structures (e.g., noise from the ventricle when recording in the atrium), power-line noise. It was noted by the present inventors that temperature-based noise was also observed on adjacent transducers (both (a) ablation-activated transducers that were activated to transmit ablative energy and (b) non-ablation-activated transducers that were not activated to transmit ablative energy, but which were within sufficient proximity to the particular ablation-activated transducer to be exposed to its ablative energy). This circumstance is also consistent with the main contributing factor of the noise being temperature-variation dependent. According to various embodiments, the temperature-based noise typically has a low frequency content (e.g., <10 Hz) and appears on some of the sensing transducers with relatively large amplitude (e.g., as shown in FIGS. 5 and 6). In some cases, the temperature-based noise may be synchronized with the patient's heartbeat, and as the heart rate changes the noise component also varies. This is believed to be because the blood flow around the sensing transducers changes throughout the cardiac cycle, and the flow variations alter the sensing transducers' temperatures. Consequently, the noise observed on the recorded electrophysiological information may vary with the heart rate. It is also noted that in some cases, the possibility of a delayed reaction may occur between the measured temperature and the electrode interface temperature due to thermal diffusion effects.

Examples of scenarios in which time-varying temperatures at or proximate a sensing transducer that may contribute to electrical noise on the measured electrophysiological activity may include, by way of non-limiting examples:
ablative energy delivery causing, for example, radiofrequency ablation, pulsed field ablation (also referred to as electroporation), or cryoablation, where the ablative energy causes temperature change at or near a sensing transducer;
energy delivered for the purpose of evaluating the thermal conditions at the device's surface for navigation or mapping purposes, such as flow-based maps (for example, as described in U.S. Pat. No. 8,920,411, issued Dec. 30, 2014 (“Gelbart et al.”) and U.S. Pat. No. 8,906,011, issued Dec. 9, 2014 (“Gelbart et al.”); and
injection of a therapeutic agent, saline, contrast agent, or other externally administered liquid that may introduce transient temperature fluctuations due to a difference in temperature between the administered liquid and the surrounding bodily fluid.

Some embodiments of the present invention pertain to systems and methods configured to measure the temperature over time at least proximate each of one or more transducers of a transducer-based device, estimate the electrical noise that may arise as a result of that time-varying temperature, and remove that noise from electrophysiological information.

FIG. 7 illustrates a programmed configuration 700 of a data processing device system (e.g., 110, 310), according to some embodiments of the present invention. For example, a programmed configuration may be implemented by the data processing device system being communicatively connected to an input-output device system (e.g., 120, 320) and a memory device system (e.g., 130, 330), and being configured by a program stored by the memory device system at least to perform one or more actions (e.g., such as at least one, more, or all of the actions described in FIG. 7 or otherwise herein). In some embodiments in which the programmed configuration illustrated in FIG. 7 actually is executed at least in part by the data processing device system, such actual execution may be considered a respective method executed by the data processing device system. In this regard, reference numeral 700 and FIG. 7 may be considered to represent one or more methods in some embodiments and, for ease of communication, one or more methods 700 may be referred to simply as method 700. The blocks shown in FIG. 7 may be associated with computer-executable instructions of a program that configures the data processing device system to perform the actions described by the respective blocks. According to various embodiments, not all of the actions or blocks shown in FIG. 7 are required, and different orderings of the actions or blocks shown in FIG. 7 may exist. In this regard, in some embodiments, a subset of the blocks shown in FIG. 7 or additional blocks may exist. In some embodiments, a different sequence of various ones of the blocks in FIG. 7 or actions described therein may exist.

In some embodiments, a memory device system (e.g., 130, 330 or a computer-readable medium system) stores the program represented by FIG. 7, and, in some embodiments, the memory device system is communicatively connected to the data processing device system as a configuration thereof. In this regard, in various example embodiments, a memory device system (e.g., memory device systems 130, 330) is communicatively connected to a data processing device system (e.g., data processing device systems 110 or 310) and stores a program executable by the data processing device system to cause the data processing device system to execute various actions described by, or otherwise associated with, the blocks illustrated in FIG. 7 for performance of some or all of method 700 via interaction with at least, for example, a transducer-based device (e.g., devices 200A, 300A, or 400A). In these various embodiments, the program may include instructions configured to perform, or cause to be performed, various ones of the block actions described by or otherwise associated with one or more or all of the blocks illustrated in FIG. 7 for performance of some, or all, of method 700.

In FIG. 7, according to some embodiments, block 702 represents a configuration of the data processing device system (e.g., data processing device systems 110 or 310) (according to a program) to acquire, generate, or receive electrophysiological information indicating sensed electrophysiological activity produced within a patient body, the electrophysiological activity sensed via at least a first transducer (e.g., 220, 306, 406). According to some embodiments, block 704 represents a configuration of the data processing device system (e.g., data processing device systems 110 or 310) (according to a program) to acquire or receive, via the input-output device system (120, 320), temperature information indicating temperature, or in some embodiments, temperature change, at least proximate the first transducer. According to some embodiments, block 706 represents a configuration of the data processing device system (e.g., data processing device systems 110 or 310) (according to a program) to modify the acquired, generated, or received electrophysiological information in accordance with the acquired or received temperature information (for example, as exemplified further below in this disclosure). According to some embodiments, block 708 represents a configuration of the data processing device system (e.g., data processing device systems 110 or 310) (according to a program) to cause output, via the input-output device system (120, 320), of the modified electrophysiological information. In some embodiments, the modified electrophysiological information is outputted to a display device system (e.g., 332).

In some embodiments, the first transducer includes an electrode (e.g., 315, 415) configured to sense electrophysiological activity. According to some embodiments, the input-output device system (120, 320) includes a transducer-based device (e.g., devices 200A, 300A, or 400A), the transducer-based device configured to deliver at least the first transducer to a bodily cavity inside the patient body. In some embodiments, the transducer-based device includes an implantable portion. According to various embodiments, the acquired, generated, or received electrophysiological information (e.g., generated or received in accordance with block 702) indicates the sensed electrophysiological activity produced within the patient body at least during a first state in which at least the first transducer is in the bodily cavity inside the patient body. In some embodiments, the acquired or received temperature information (e.g., received in accordance with block 704) indicates the temperature at least proximate the first transducer at least during the first state in which at least the first transducer is in the bodily cavity inside the patient body. In some embodiments, in which at least the first transducer is in the bodily cavity inside the patient body, the acquired, generated, or received electrophysiological information may take the form of an electrogram. In some embodiments, in which at least the first transducer is in the bodily cavity inside the patient body, the outputted modified electrophysiological information may take the form of an electrogram.

Referring back to Block 704, the temperature information indicating temperature or temperature change at least proximate the first transducer may be received from a particular transducer other than the first transducer according to some embodiments. For example, in some embodiments, a first transducer may sense electrophysiological activity, while another transducer may sense temperature or temperature change at least proximate the first transducer. In some embodiments, the temperature information indicating temperature or temperature change at least proximate the first transducer may be received from a particular transducer set of a transducer-based device (e.g., 200A, 300A, 400A) that includes the first particular transducer. For example, in some embodiments a first transducer may sense electrophysiological activity, and the first transducer and possibly one or more other transducers may sense temperature or temperature change at least proximate the first transducer. According to some embodiments, the first transducer may be configured to sense temperature, and the temperature information indicating the temperature at least proximate the first transducer is sensed via the first transducer. For example, in FIG. 4, each of the transducers 406 includes a respective temperature sensor 408. Whether or not the temperature information indicating the temperature at least proximate the first transducer is sensed via the first transducer or some other transducer may be motivated by the specific capability of the first transducer, the proximity of other transducers, or the specific capability of the other transducers. In some embodiments, the first transducer includes (a) an electrode configured to sense the electrophysiological activity, and (b) a thermal sensor responsive to the temperature at least proximate the first transducer. For example, in FIG. 4, each transducer 406 includes an electrode 415 configured to, among other things, sense electrophysiological information, and each transducer 406 further includes a thermal sensor 408, according to some embodiments. In some embodiments, a fluid (e.g., a therapeutic agent, saline, contrast agent, or other externally administered liquid) is delivered to the bodily cavity in the patient body, and a temperature change at least proximate the first transducer is responsive to the delivered fluid.

According to some embodiments, the data processing device system (110, 310) is configured at least by the program at least to cause the transducer-based device (e.g., 200A, 300A, 400A), or at least part of the transducer-based device (e.g., 200A, 300A, 400A), to transmit energy. In some embodiments, such transmission of energy may occur just before or during execution of block 702 in FIG. 7. In some embodiments, the temperature information indicating the temperature or temperature change at least proximate the first transducer is responsive to the energy transmitted by the transducer-based device (e.g., 200A, 300A, 400A) or the part of the transducer-based device (e.g., 200A, 300A, 400A) in the bodily cavity at least during the first state in which at least the first transducer is in the bodily cavity inside the patient body. The transducer-based device, or the part of the transducer-based device can take different forms according to various embodiments. The energy transmitted by the transducer-based device or the part of the transducer-based device can take different forms according to various embodiments. For example, in some embodiments, the transducer-based device or the part of the transducer-based device can transmit energy in accordance with various functions associated with the transducer-based device.

For instance, in some embodiments, the transducer-based device (e.g., 200A, 300A, 400A) may be delivered into the bodily cavity in the patient body, and the data processing device system (110, 310) may be configured to determine a level of contact between at least part of the transducer-based device and a tissue surface of the bodily cavity in the patient body. In some of these embodiments, the energy may be transmitted at least as part of the determining the level of contact between at least part of the transducer-based device and the tissue surface of the bodily cavity in the patient body. In some embodiments, the transducer-based device or the part of the transducer-based device may be activated to transmit energy to cause tissue stimulation or to pace a cardiac cycle at a particular rate. In some embodiments, the transducer-based device or the part of the transducer-based device may be activated to transmit energy to determine a tissue electrical characteristic (e.g., impedance or permittivity). In some embodiments, the transducer-based or the part of the transducer-based device (e.g., 200A, 300A, 400A) may be activated to transmit energy to determine if a shunting condition may be associated with a portion of the transducer-based device (e.g., 200A, 300A, 400A) that may hinder a desired functioning of the transducer-based device. See, e.g., U.S. Pat. No. 10,792,089, issued Oct. 6, 2020 (“Goertzen et al.”) regarding the transmission of energy to determine if a shunting condition exists. In some embodiments, the transducer-based device (e.g., 200A, 300A, 400A) or the part of the transducer-based device (e.g., 200A, 300A, 400A) may be activated to transmit energy as part of a navigation process determining a location, an orientation, or both a location and an orientation of a portion of the transducer-based device in the bodily cavity. In some embodiments, the transducer-based device (e.g., 200A, 300A, 400A) or the part of the transducer-based device (e.g., 200A, 300A, 400A) may be activated to transmit energy as part of a mapping process that maps various anatomical features in a bodily cavity (for example, as described in U.S. Pat. No. 8,920,411, issued Dec. 30, 2014 (“Gelbart et al.”) and U.S. Pat. No. 8,906,011, issued Dec. 9, 2014 (“Gelbart et al”). In this regard, it can be seen that, in some embodiments, the transmitted energy may be insufficient for tissue ablation. However, in some embodiments, the transmitted energy may be configured to cause tissue ablation. According to various embodiments, the energy transmitted by the transducer-based device (e.g., 200A, 300A, 400A) or the part of the transducer-based device (e.g., 200A, 300A, 400A) may be sufficient to cause temperatures changes at least proximate the first transducer. As discussed above, such temperature changes may introduce time-varying noise into sensed electrophysiological activity, which may then be reduced according to various embodiments of the present invention.

In some embodiments, the part of the transducer-based device (e.g., 200A, 300A, 400A) includes a second transducer (e.g., 220, 306, 406) configured to transmit the energy. In some embodiments, the second transducer is the first transducer, such that, for example, the same transducer that senses electrophysiological activity may also sense temperature. In this regard, in some embodiments, the first transducer both transmits the energy and senses the temperature information indicating temperature at least proximate the first transducer. In some embodiments, the second transducer is other than the first transducer. In some embodiments, the second transducer is a particular transducer positioned at least proximate the first transducer. In some embodiments, the second transducer transmits the energy and the first transducer senses the temperature information indicating temperature at least proximate the first transducer. In some embodiments, the second transducer and the first transducer are adjacent transducers.

According to some embodiments, as discussed above, the energy is sufficient to ablate tissue in the bodily cavity. In this regard, in some embodiments, the first transducer is configured to transmit tissue ablative energy. According to some embodiments, the energy is sufficient to thermally ablate tissue in the bodily cavity. Thermal ablation can take different forms according to various embodiments. For example, the data processing device system (110, 310) may be configured at least by the program at least to cause, via the transducer-based device (200A, 300A, 400A) communicatively connected to the input-output device system (120, 320), radiofrequency (“RF”) ablation of tissue in the bodily cavity in the patient body, and the energy is transmitted at least as part of the causing RF ablation of tissue in the bodily cavity in the patient body. In some embodiments, the data processing device system (110, 310) may be configured at least by the program at least to cause, via a cryogenic device communicatively connected to the input-output device system (120, 320), cryoablation of tissue in the bodily cavity in the patient body, and the energy is transmitted at least as part of the causing cryoablation of tissue in the bodily cavity in the patient body. In some embodiments, the data processing device system (110, 310) may be configured at least by the program at least to cause, via the transducer-based device (200A, 300A, 400A) communicatively connected to the input-output device system (120, 320), pulsed field ablation of tissue in the bodily cavity in the patient body, wherein the energy is transmitted at least as part of the causing pulsed field ablation of tissue in the bodily cavity in the patient body.

In some embodiments, the first transducer includes a first electrode (e.g., 315, 415) that includes a first conductive metal (for example, a surface portion such as first transmission surface 319 made from the first conductive metal). In some embodiments, the input-output device system (120, 320) includes a second electrode that includes a second conductive metal (for example, a surface portion made from the second conductive metal). Different metals will have different degrees of conductivity. For example, tungsten and bismuth are considered to be generally poor conductors of electricity. Stainless steel is also considered to be a relatively poor conductor of electricity due to its alloy structure. Gold, platinum, copper and silver tend to have excellent electrical conductive properties. Electrodes employing gold or platinum energy transmission surfaces (319) tend to be used when employed inside the body due to biocompatibility requirements. In some embodiments, the data processing device system (110, 310) is configured at least by the program at least to generate or receive the electrophysiological information indicating the electrophysiological activity generated within the patient body based at least on intracardiac voltage data indicating electric potential between the first electrode and the second electrode. In some embodiments, the second metal is different than the first metal. In some embodiments, the second electrode is configured to be externally applied to the patient body that includes the bodily cavity (for example, indifferent electrode 326).

According to some embodiments, the data processing device system (e.g., 110, 310) may be configured at least by the program (e.g., program instructions associated with block 706 in FIG. 7, in some embodiments) at least to produce the modified electrophysiological information in accordance with the acquired or received temperature information (e.g., received per block 704, in some embodiments) to mitigate noise in at least the electrophysiological information (generated or received per block 702, in some embodiments) caused by thermoelectric effects (e.g., the Seebeck effect described above in this disclosure) during measurement of the electrophysiological information (which may in some embodiments, be the electric potential measured between the above-discussed first electrode and the second electrode). In some embodiments, the data processing device system (e.g., 110, 310) may be configured at least by the program at least to produce the modified electrophysiological information in accordance with the acquired or received temperature information to mitigate noise in at least the intracardiac voltage data caused at least in part by a temperature difference between the first electrode and the second electrode during measurement of the electric potential between the first electrode and the second electrode. In some embodiments, the data processing device system (e.g., 110, 310) may be configured at least by the program at least to produce the modified electrophysiological information in accordance with the acquired or received temperature information to mitigate noise in at least the intracardiac voltage data caused by temperature-dependent galvanic cell potential effects during measurement of the electric potential between the first electrode and the second electrode. A galvanic or voltaic cell is an electrochemical cell in which current is generated from spontaneous redox reactions. Galvanic cell potential effects may occur when the first electrode includes, or is made up of, a different metal than the second electrode.

Referring back to FIG. 7, the data processing device system (e.g., 110, 310) may be, according to some embodiments, configured at least by the program (e.g., instructions associated with block 706, in some embodiments) at least to, in accordance with the acquired or received temperature information, (a) determine an amount of change in temperature at least proximate the first transducer (e.g., based on information received according to block 704), and (b) determine an amount of modification of the acquired, generated, or received electrophysiological information (e.g., generated or received per block 702) based at least on the determined amount of change in temperature at least proximate the first transducer. According to some embodiments, the data processing device system (110, 310) may be configured at least by the program at least to produce the modified electrophysiological information in accordance with the determined amount of modification. According to some embodiments, the data processing device system (110, 310) may be configured at least by the program at least to produce the modified electrophysiological information at least in part by subtracting the determined amount of modification from the acquired, generated, or received electrophysiological information. In this context, the word “subtracting” is to be viewed a mathematical operation configured to reduce noise in the acquired, generated, or received electrophysiological information. Any mathematical operation that operates in this capacity is considered in this disclosure to be analogous to “subtracting”.

In some embodiments, the temperature information indicates temperature at least proximate the first transducer over, during, or within a time interval, and the acquired, generated, or received electrophysiological information indicates an electrophysiological information waveform representing the sensed electrophysiological activity produced within the patient body and sensed via at least the first transducer over, during, or within the time interval. According to some embodiments, the data processing device system (110, 310) may be configured at least by the program (e.g., via instructions associated with block 704) at least to generate a temperature-based information waveform representing the temperature at least proximate the first transducer over, during, or within the time interval indicated by the temperature information. In some embodiments, the data processing device system 110, 310 may be configured at least by the program at least to produce the modified electrophysiological information (e.g., per block 706) at least in part by subtracting the temperature-based information waveform from the electrophysiological information waveform.

According to some embodiments, the temperature information may indicate temperature at least proximate the first transducer over, during, or within, a time interval. In some embodiments, the data processing device system 110, 310 may be configured at least by the program at least to, in accordance with the acquired or received temperature information (e.g., acquired or received per block 704), (a) determine an amount of change in temperature at least proximate the first transducer, and (b) determine an amount of modification of the acquired, generated, or received electrophysiological information based at least on the determined amount of change in temperature at least proximate the first transducer. In some embodiments, the data processing device system 110, 310 may be configured at least by the program at least to produce the modified electrophysiological information (e.g., per block 706) in accordance with the acquired or received temperature information in accordance with the determined amount of modification. In some embodiments, the electrophysiological information indicates the sensed electrophysiological activity produced within the patient body over, during, or within the time interval. In some embodiments, the electrophysiological activity produced in the patient body is sensed during the time interval.

According to various embodiments, the data processing device system (110, 310) may be configured at least by the program at least to determine the amount of the modification based at least in part via a linear mapping of the temperature information (for example, as described in further detail below). In some embodiments, the data processing device system (110, 310) may be configured at least by the program at least to determine the amount of the modification based at least in part via a linear mapping over time of the temperature information. For example, in some embodiments, temperature-based noise may be reduced by removing the similarities between the temperature and unfiltered data representative of the electrophysiological information (e.g., an unfiltered electrogram). According to some embodiments, the temperature may be mathematically transformed (e.g., via a linear transformation or mapping) to become similar to the unfiltered data representative of the electrophysiological information (for example, as described below in this disclosure). According to some embodiments, this similarity is subtracted from the data representative of the electrophysiological information thereby canceling or reducing the temperature-based noise. According to various embodiments, the employed linear transformation may be considered in keeping with a use of the Nernst equation which predicts a linear relation between temperature and cell potential.

According to some embodiments, the present inventors recognized that determining the temperature-based noise within the electrophysiological activity data may be achieved as the solution to a least squares problem. According to some embodiments, a series of parameters relating the measured temperature to the resulting electrophysiological activity noise may be found. According to various embodiments, the found parameters may estimate the electrophysiological activity noise. According to some embodiments, the estimated electrophysiological activity noise is subtracted from the electrophysiological activity data to produce modified electrophysiological information with reduced noise. According to some embodiments, the parameters may include a temperature scaling factor, s, additive component, c, and a delay component between voltage and temperature Δ. The following equations establish a relationship between the sampled temperature data (e.g., sampled per block 704, in some embodiments) and the sampled electrophysiological voltages (e.g., sampled per block 702, in some embodiments) that may be employed according to various embodiments to achieve the modified electrophysiological information per block 706. In this regard, an N-dimensional vector v made up of N samples of the unfiltered electrophysiological voltage data is employed. For a value of N (e.g., a realtively small value of N such that the length of data represented (i.e., N * sampling period) may be on the order of a few seconds, (e.g., 0.25 s-5.0 s) in some embodiments, while other values of N may be employed in other embodiments), the sampled electrophysiological voltages may be expressed as:

v[n]=d[n]+s[n]t[n−Δ]−c[n]1_{N}+∈[n] (4)

where each term in equation (4) is defined as follows:

$$v [ n ] = ( \begin{matrix}v [ n ] \\ v [ n - 1 ] \\ ⋮ \\ v [ n - N + 1 ]\end{matrix} )$$

is a vector of the last N samples of measured electrophysiological voltage;

$$d [ n ] = ( \begin{matrix}d [ n ] \\ d [ n - 1 ] \\ ⋮ \\ d [ n - N + 1 ]\end{matrix} )$$

is a vector of the last N samples of the “desired” signal (e.g., the modified electrophysiological information per block 706, in some embodiments), which is the desired electrophysiological voltage with the determined noise removed;

s[n] is a temperature related scaling factor;

$$t [ n - Δ ] = ( \begin{matrix}t [ n - Δ ] \\ t [ n - Δ - 1 ] \\ ⋮ \\ t [ n - Δ - N + 1 ]\end{matrix} )$$

is a vector of length N describing measured temperature subject to a delay parameter Δ (In some cases there may be a delay between the measured temperature information and the recorded electrogram. For instance, an electrode may measure voltage virtually instantaneous while a temperature sensor may have some delay associated with it. If the temperature measurements are delayed by, for example, 0.25 second relative to the voltage measurements, it may, in some embodiments, be preferable to determine corrections for voltage(t) at t+0.25 seconds, since this may be a better indicator of the temperature that was actually contributing to the voltage noise. The same is also true of delays due to processing/data transmission. This delay parameter Δ is employed to take that effect into account. In some embodiments, Δ may be “integer-valued” so that it corresponds to a discrete sample. In some embodiments, Δ may be “non-integer valued” (for example, if interpolation is employed). Δ may be a positive or negative value);

c[n] is an estimate of the constant factor, which represents a combination of a temperature related constant factor and the baseline wander of the electrogram;

1_{N} is an N-dimensional vector with all elements equal to 1;

∈[n] is N-dimensional vector of random noise (e.g., unrelated to temperature); Based on the model (3), the measured electrophysiological potential is equal to the summation of the following terms:
a) the true or desired underlying electrophysiological potential, d[n];
b) a DC component c[n] which represents the baseline wander and other slowly varying or fixed components;
c) a temperature-related noise component, s[n]t[n−Δ] which is linearly related to the measured temperature after accounting for delay between the two signals, Δ; and
d)∈[n] which represents other noise components (e.g., measurement noise, modeling error).

According to some embodiments, parameter Δ may be used in equation (4) to represent the delay between the sampled electrophysiological voltage (e.g., sampled per block 702, in some embodiments) and the sampled temperature (e.g., sampled per block 704, in some embodiments). One reason for the presence of delay between the sampled electrophysiological voltage and the sampled temperature may be associated with non-simultaneous temperature and voltage measurements, or thermal conduction delay effects. Another contributing factor may be the temporal response of an underlying chemical phenomena. It is noted that in equation (4), the choice of applying the delay term Δ to temperature rather than the electrophysiological voltage is arbitrary, with the sign of the delay indicating which signal is leading and which is lagging.

According to various embodiments, the “desired” signal d[n] nay be determined by solving for values of s, Δ, and c in the following least squares formulation:

$${min}_{s [ n ] , c [ n ] , Δ} {‖ v [ n ] - s [ n ] t [ n - Δ ] + c [ n ] {1}_{N} ‖}^{2} \qquad ( 5 )$$

In some embodiments (for example, where it is desired to manage computational and algorithmic complexity), Δ may instead be a fixed value determined based on an offline characterization of the above optimization when performed in post-processing. Given a fixed value of Δ, the optimum values of s and c are given by:

$$s = \frac{\overline{tv} - \overline{t} \overline{v}}{\overline{{t}^{2}} - {( \overline{t} )}^{2}} = \frac{x cov ( t , v )}{var ( t )} and c = s \overline{t} - \overline{v} \qquad ( 6 )$$

It is noted that, in this context, a “bar” over a variable indicates averaging and N indicates the length of the window in time over which the minimization is performed. According to various embodiments, at each timepoint or sample, n, the above optimization is performed on temperature and electrophysiological voltage measurements in the range of [n−N+1, n] to determine estimates of parameters s and c, s′[n] and c′[n]. An estimate of the true electrogram signal, d′[n], is then calculated according to:

d′[n]=v[n]−s′[n]t[n−Δ]+c′[n] (7)

It is noted that, according to various embodiments, the temperature sampling rate is configured to match, or be close to, the electrophysiological activity sampling rate to achieve relatively more favorable results. However, when fast temperature changes are not present, relatively lower temperature sampling rates may be employed effectively. In some cases, the present inventors have employed electrophysiological activity sampling rates (e.g., electrogram sampling rates) that were 49 times higher than the temperature sampling rate with good results. In this regard, in order to have a temperature value with every electrophysiological activity sample, the present inventors up-sampled the temperature data using linear interpolation by a factor of 49. Consequently, after the interpolation, the sampling frequencies of the temperature and the electrophysiological activity were considered to be essentially identical (e.g., 488.2813 Hz in this case). Thereafter, for every sample, n, the present inventors were able to derive a temperature value, t[n], and measured electrophysiological voltage, v[n].

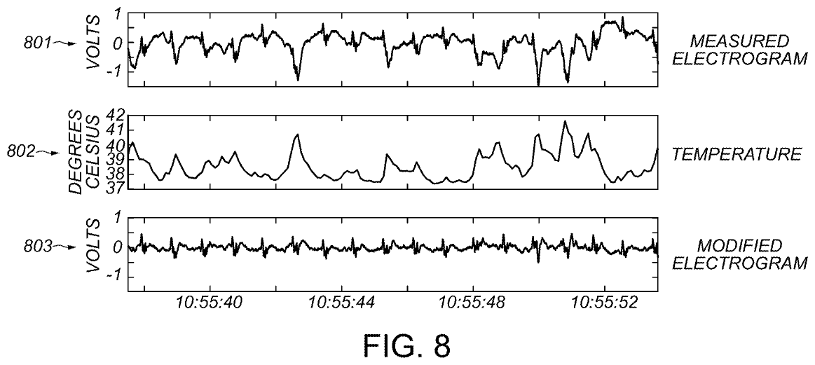

FIG. 8 shows, among other things, sample output of the method described above with respect to equation (7), which may be a particular implementation of block 706, according to some embodiments. FIG. 8, row 801 shows measured electrophysiological voltage data (e.g., an electrogram in this embodiment), which may correspond to measured electrophysiological voltage data according to block 702 in FIG. 7, according to some embodiments. Row 802 shows measured temperature data, which may correspond to measured temperature data according to block 704 in FIG. 7, according to some embodiments. In this example of FIG. 8, both the electrophysiological voltage data (row 801) and the measured temperature data (row 802) were recorded by a particular transducer (similar in form and function to transducer 406), the particular transducer positioned proximately to a plurality of transducers activated to transmit ablative energy (i.e., RF ablative energy in this case). Due to the proximity of the particular transducer to the ablating transducers, the temperature measured by the particular transducer fluctuated. The plot of the electrophysiological voltage in row 801 displays various artifacts, some having relatively large amplitude fluctuations. It is noted that the large fluctuation in the electrophysiological voltage plot correspond to various large fluctuations in the temperature plot in row 802. Row 803 shows the modified electrophysiological voltage (e.g., per block 706 in FIG. 7, also in the form of an electrogram) after the method associated with equation (7) is used to remove the temperature-based noise. The plot of the modified electrophysiological voltage in row 803 shows a “classic” electrogram form with significantly fewer and smaller artifacts. This example confirms that the methods described in this disclosure can properly remove the temperature-based noise from electrophysiological voltage data. The modified electrophysiological voltage in the form of an electrogram in row 803 may be visually presented to a user via a display device system (e.g., 332) per block 708 in FIG. 7, although other forms of output may be implemented in other embodiments.

While some of the embodiments of processing electrophysiological information disclosed above are described with examples of cardiac mapping, ablation, or both, the same or similar embodiments may be employed in conjunction with other applications involving mapping, ablating, or both, of other bodily organs, for example with respect to the brain, or any bodily organ to which the devices of the present invention may be introduced.

Subsets or combinations of various embodiments described above can provide further embodiments.

These and other changes can be made to the invention in light of the above-detailed description. In general, in the following claims, the terms used should not be construed to limit the invention to the specific embodiments disclosed in the specification and the claims, but should be construed to include other transducer-based device systems including all medical treatment device systems and all medical diagnostic device systems in accordance with the claims. Accordingly, the invention is not limited by the disclosure.

What is claimed is:

1. An electrophysiological information processing system comprising:
an input-output device system;
a memory device system storing a program; and
a data processing device system communicatively connected to the input-output device system and the memory device system, the data processing device system configured at least by the program at least to:
receive electrophysiological information indicating sensed electrophysiological activity produced within a patient body, the electrophysiological activity sensed via at least a first transducer;
receive, via the input-output device system, temperature information indicating temperature at least proximate the first transducer;
modify the received electrophysiological information in accordance with the received temperature information; and
cause output, via the input-output device system, of the modified electrophysiological information.

2. The electrophysiological information processing system of claim 1,
wherein the input-output device system comprises a transducer-based device, the transducer-based device configured to deliver at least the first transducer to a bodily cavity inside the patient body,
wherein the received electrophysiological information indicates the sensed electrophysiological activity produced within the patient body at least during a first state in which at least the first transducer is in the bodily cavity inside the patient body, and
wherein the received temperature information indicates the temperature at least proximate the first transducer at least during the first state in which at least the first transducer is in the bodily cavity inside the patient body.

3. The electrophysiological information processing system of claim 1, wherein the first transducer is configured to sense temperature, and wherein the temperature information indicating the temperature at least proximate the first transducer is sensed via the first transducer.

4. The electrophysiological information processing system of claim 3, wherein the first transducer comprises an electrode configured to sense the electrophysiological activity, and comprises a thermal sensor responsive to the temperature at least proximate the first transducer.

5. The electrophysiological information processing system of claim 2,
wherein the data processing device system is configured at least by the program at least to cause at least part of the transducer-based device to transmit energy, and
wherein the received temperature information indicating the temperature at least proximate the first transducer is responsive to the energy transmitted by the at least part of the transducer-based device in the bodily cavity at least during the first state in which at least the first transducer is in the bodily cavity inside the patient body.

6. The electrophysiological information processing system of claim 5, wherein the part of the transducer-based device comprises a second transducer configured to transmit the energy.

7. The electrophysiological information processing system of claim 5, wherein the energy is sufficient to ablate tissue in the bodily cavity.

8. The electrophysiological information processing system of claim 5, wherein the energy is configured to thermally ablate tissue in the bodily cavity.

9. The electrophysiological information processing system of claim 2, wherein the first transducer is configured to transmit tissue ablative energy.

10. The electrophysiological information processing system of claim 2,
wherein the first transducer comprises a first electrode comprising a first conductive metal,
wherein the input-output device system comprises a second electrode comprising a second metal, and
wherein the received electrophysiological information indicating the sensed electrophysiological activity produced within the patient body is based at least on intracardiac voltage data indicating electric potential between the first electrode and the second electrode.

11. The electrophysiological information processing system of claim 10, wherein the second electrode is configured to be externally applied to the patient body comprising the bodily cavity.

12. The electrophysiological information processing system of claim 10, wherein the data processing device system is configured at least by the program at least to produce the modified electrophysiological information in accordance with the received temperature information to mitigate noise in at least the intracardiac voltage data caused by thermoelectric effects during measurement of the electric potential between the first electrode and the second electrode.

13. The electrophysiological information processing system of claim 10, wherein the data processing device system is configured at least by the program at least to produce the modified electrophysiological information in accordance with the received temperature information to mitigate noise in at least the intracardiac voltage data caused at least in part by a temperature difference between the first electrode and the second electrode during measurement of the electric potential between the first electrode and the second electrode.

14. The electrophysiological information processing system of claim 10, wherein the second metal is different than the first metal.

15. The electrophysiological information processing system of claim 10, wherein the data processing device system is configured at least by the program at least to produce the modified electrophysiological information in accordance with the received temperature information to mitigate noise in at least the intracardiac voltage data caused by temperature-dependent galvanic cell potential effects during measurement of the electric potential between the first electrode and the second electrode.

16. The electrophysiological information processing system of claim 1,
wherein the temperature information indicates temperature at least proximate the first transducer during a time interval,
wherein the data processing device system is configured at least by the program at least to, in accordance with the received temperature information, (a) determine an amount of change in temperature at least proximate the first transducer, and (b) determine an amount of modification of the received electrophysiological information based at least on the determined amount of change in temperature at least proximate the first transducer, and
wherein the data processing device system is configured at least by the program at least to produce the modified electrophysiological information in accordance with the received temperature information in accordance with the determined amount of modification.

17. The electrophysiological information processing device system of claim 16, wherein the electrophysiological information indicates the sensed electrophysiological activity produced within the patient body during the time interval.

18. The electrophysiological information processing system of claim 1,
wherein the data processing device system is configured at least by the program at least to, in accordance with the received temperature information, (a) determine an amount of change in temperature at least proximate the first transducer, and (b) determine an amount of modification of the received electrophysiological information based at least on the determined amount of change in temperature at least proximate the first transducer, and
wherein the data processing device system is configured at least by the program at least to produce the modified electrophysiological information at least in part by subtracting the determined amount of modification from the received electrophysiological information.

19. The electrophysiological information processing system of claim 1,
wherein the temperature information indicates temperature at least proximate the first transducer during a time interval,
wherein the received electrophysiological information indicates an electrophysiological information waveform representing the sensed electrophysiological activity produced within the patient body and sensed via at least the first transducer during the time interval,
wherein the data processing device system is configured at least by the program at least to generate a temperature-based information waveform representing the temperature at least proximate the first transducer during the time interval indicated by the temperature information, and
wherein the data processing device system is configured at least by the program at least to produce the modified electrophysiological information at least in part by subtracting the temperature-based information waveform from the electrophysiological information waveform.

20. The electrophysiological information processing system of claim 16, wherein the data processing device system is configured at least by the program at least to determine the amount of the modification based at least in part via a linear mapping of the temperature information.

21. The electrophysiological information processing system of claim 16, wherein the data processing device system is configured at least by the program at least to determine the amount of the modification based at least in part via a linear mapping over time of the temperature information.

22. A method of processing electrophysiological information, the method executed by a data processing device system configured by a program, the data processing device system communicatively connected to an input-output device system and a memory device system, the memory device system storing the program, and the method comprising:
receiving electrophysiological information indicating sensed electrophysiological activity produced within a patient body, the electrophysiological activity sensed via at least a first transducer;
receiving, via the input-output device system, temperature information indicating temperature at least proximate the first transducer;
modifying the received electrophysiological information in accordance with the received temperature information; and
causing output, via the input-output device system, of the modified electrophysiological information.

23. A method of processing electrophysiological information, the method executed by a data processing device system configured by a program, the data processing device system communicatively connected to an input-output device system and a memory device system, the memory device system storing the program, and the method comprising:
acquiring electrophysiological information indicating sensed electrophysiological activity produced in a patient body, the electrophysiological activity sensed via at least a first transducer;
acquiring temperature information indicating temperature change at least proximate the first transducer;
modifying the electrophysiological information in accordance with the acquired temperature information; and
causing output, via the input-output device system, of the modified electrophysiological information.
